(19) United States
(12) Patent Application Publication
(10) Pub. No.: US 2026/0042735 A1
(43) Pub. Date: Feb. 12, 2026

(54) BENZYLTRYPTAMINE COMPOUNDS

(71) Applicant: Reunion Neuroscience, Inc., Morristown, NJ (US)

(72) Inventor: Nathan Bryson, Toronto (CA)

(21) Appl. No.: 19/094,736

(22) Filed: Mar. 28, 2025

Related U.S. Application Data
(63) Continuation of application No. 18/644,754, filed on Apr. 24, 2024, now abandoned.
Application No. 18/644,754 is a continuation of application No. 17/977,398, filed on Oct. 31, 2022, now abandoned.
(60) Provisional application No. 63/273,720, filed on Oct. 29, 2021.
(60) Provisional application No. 63/334,443, filed on Apr. 25, 2022.

(51) Int. Cl. C07D 209/14; A61P 25/24

(52) U.S. Cl. CPC C07D 209/14; A61P 25/24

(57) ABSTRACT

There is disclosed a compound of Formula (I):

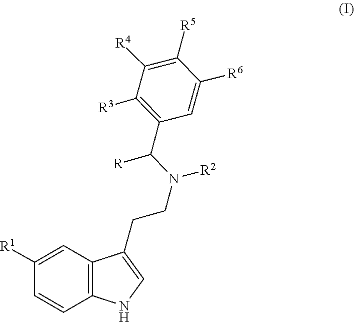

and any pharmaceutically acceptable salt or zwitterion thereof, wherein: R is hydrogen, methyl or ethyl; R¹ is hydrogen or C₁-C₂ alkoxy; R² is methyl or a C₂-C₄ group which may be saturated or unsaturated, branched or linear; and R³, R⁴, R⁵ and R⁶ each are independently selected from hydrogen, hydroxyl, halogen, methyl optionally substituted with hydroxy, methoxy, ethoxy, and a saturated or unsaturated C₂-C₃ that may be optionally substituted with hydroxyl, with the provisos that: (i) at least two of R⁴, R⁵, R⁶ and R⁷ must be hydrogen, and (ii) R³, R⁴, R⁵ and R⁶ may be selected such that an adjacent pair thereof join to form a ring having at least 5 members. The compound of Formula (I) is believed useful in treating a disease or disorder in a subject which may be alleviated by a 5HT2A agonist (e.g., CNS disorders and one or more symptoms of any one of depression, alcoholism, tobacco addiction, cocaine addiction, inflammation, cluster headache and PTSD in a subject).

### CROSS-REFERENCE TO RELATED APPLICATION

The present application claims priority under 35 U.S.C. § 119(e) to U.S. Provisional Patent Application No. 63/273,720, filed Oct. 29, 2021, and entitled “Novel N-Methoxybenzyltryptamine Compound” and to U.S. Provisional Patent Application No. 63/334,443, filed Apr. 25, 2022, and entitled “Novel N-Methoxybenzyltryptamine Compound” which are each hereby incorporated by reference in their entireties.

### BACKGROUND OF THE INVENTION

### Field of the Invention

In one of its aspects, the present disclosure relates to a novel benzyl-tryptamine compound, preferably a novel N-methoxybenzyl-tryptamine compound. In another of its aspects, the present disclosure relates to process for making a benzyl-tryptamine compound, preferably a N-methoxybenzyl-tryptamine compound. In yet another of its aspects, the present disclosure relates to a novel pharmaceutical composition. In yet another of its aspects, the present disclosure relates to treatment of a CNS disorder.

### Description of the Prior Art

Psilocybin is a naturally occurring psychedelic prodrug compound produced by more than 200 species of mushrooms which are collectively known as psilocybin mushrooms. As a prodrug, psilocybin is quickly metabolized by the body to generate the bioactive compound psilocin, which has mind-altering effects not unlike those produced by lysergic acid diethylamide) LSD, mescaline and N,N-dimethyltryptamine (DMT). These effects include, inter alia, euphoria, visual and mental hallucinations, changes in perception, a distorted sense of time, and spiritual experiences, and can also include possible adverse reactions such as nausea and panic attacks.

While psilocybin and its therapeutic potential, along with that of other psychedelic drugs like LSD in psychiatry, was recognized and explored over 50 years ago by Hofmann and co workers at Sandoz (see, for example, Hofmann, A., Troxler, F. U.S. Pat. Nos. 3,075,992 and 3,078,214), subsequent investigation into the recreational use of psilocybin and related psychedelic drugs (e.g., LSD) was curtailed in the early 1970s. Since then, psilocybin remains classified as a scheduled drug of abuse in most countries by many national drug laws.

However, clinical investigations have recently led to increased awareness of the potential for psychedelic drugs and psilocybin in particular as breakthrough therapies to treat CNS diseases of unmet medical need. These diseases that may be addressed include both difficult to treat mental health disorders (Daniel J, Haberman M. Clinical potential of psilocybin as a treatment for mental health conditions. Ment. Health Clin. 2017, 7(1), 24-8) associated with significant morbidity such as treatment resistant depression (TRD), along with alcoholism and cocaine and tobacco addiction, as well as neurological disorders such as cluster headache which also has significant associated morbidity.

As a phosphate phenolic prodrug, psilocybin, once ingested is rapidly metabolized to the bioactive constituent psilocin, which then acts on serotonin receptors in the brain.

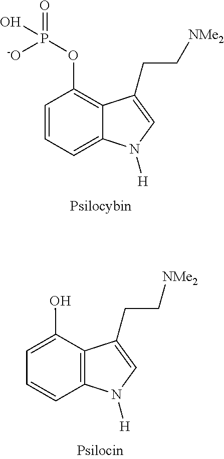

The 5-hydroxytryptamine receptors (5-HT) receptors, or serotonin receptors, are a group of G protein-coupled receptors and ligand-gated ion channels found in both the central and peripheral nervous systems. They mediate both excitatory and inhibitory neurotransmission. The 5-HT receptors are activated by the neurotransmitter 5-hydroxytryptamine more commonly known as serotonin, which is the natural ligand. 100081 Other prodrug linkages are taught in the literature, such as Wiemer et al. Top Curr Chem. 2015; 360: 115-160 and Mahato et al. Adv Drug Deliv Rev. 2011 Jul. 18; 63(8): 659-670.

As a novel antidepressant, in a small clinical trial involving cancer patients treated with psilocybin, it was found that high-dose psilocybin produced large decreases in clinician- and self-rated measures of depressed mood and anxiety, along with increases in quality of life, life meaning, and optimism, and decreases in death anxiety. In a 6-month follow-up, these changes were sustained, with about 80% of participants continuing to show clinically significant decreases in depressed mood and anxiety.

In a proof-of-concept study for psilocybin for use in treating alcohol dependence, and that involved 10 patients with a diagnosis of alcohol dependence per the DSM-IV, a significant decrease in alcohol use post psilocybin administration among the patients was observed (Bogenschutz M P, et al., Psilocybin-assisted treatment for alcohol dependence: a proof-of-concept study. J. Psychopharmacol. 2015, 29(3), 289-99).

In a study on the treatment of tobacco addiction with psilocybin, researchers found that eighty percent of participants showed biologically verified 7-day point-prevalence abstinence at 6-month follow up. The 12-month follow-up showed 67% of the participants were biologically verified as being abstinent. A follow up 2.5 years after the target quit date showed 75% were abstinent (Johnson, M. W., and Griffiths, R. R. (2017) Potential Therapeutic Effects of Psilocybin. Neurotherapeutics. 14, 734).

Pharmacologically, the bioactive constituent psilocin has been shown to act at a number of serotonin receptor subtypes from in vitro receptor binding and functional assays as summarized in Table 1 below, which is in line with its structural resemblance to serotonin (Geiger H. A., Wurst M. G., Daniels R. N., “DARK Classics in Chemical Neuroscience: Psilocybin,” ACS Chem. Neurosci. 2018, 9 (10), 2438-2447).

TABLE 1
Pharmacological Properties of Psilocin

| Binding Site | K₁ (nM) and EC₅₀ Values |
|---|---|
| SERT | 3801 |
| 5-HT1A | 567 |
| 5-HT1B | 219 |
| 5-HT1D | 36 |
| 5-HT2A | 107; EC₅₀ = 24 (43%) |
| 5-HT2B | 4.6; EC₅₀ = 58 (45%) |
| 5-HT2C | 97; EC₅₀ = 12 (45%) |
| 5-HT3 | >10000 |
| 5-HT5 | 84 |
| 5-HT6 | 57 |
| 5-HT7 | 3.5 |

EC₅₀ values for activation of PI hydrolysis in cells expressing human receptors relative to serotonin at 100%. Efficacy is provided in parentheses.

Psilocin exhibited no significant effect on dopamine receptors (unlike LSD) and appears to only act upon the noradrenergic system at very high dosages. The diverse pharmacological effects of particular relevance to therapeutic utility and limitations can be ascribed to psilocin's activation of 5-HT2A, 5-HT2B, and 5-HT2C receptors specifically as a functional agonist.

Receptor binding assays are used to characterize the interaction between a receptor molecule and any potential ligands. Such assays can determine the intrinsic affinity of ligands to the receptor, association/dissociation rates, and also the density of receptor in tissues or cells. Receptor binding assay is typically a cell-free method suitable for many GPCR (5HT receptors are G-Protein Coupled Receptors) screening that does not involve downstream signaling from the receptor. This type of assay cannot distinguish whether the candidate compound is an agonist, antagonist, or inverse agonist, only whether it binds to the receptor. The analysis of the biological responses after compound binding requires functional assays. Upon ligand binding, GPCRs change their conformation and activate coupled G proteins, which subsequently promote second messenger production via downstream effectors. Functional assays measure either G protein activation or G protein-mediated events, including second messenger generation and reporter activation, are therefore defined as G-protein-dependent functional assays. (Zhang R, Xie X Acta Pharamaca Sinica 2012, 33,372).

A known limitation to the therapeutic potential for psilocybin is a serious toxicological safety liability, namely cardiac valvulopathy, that can be anticipated from psilocin's potent agonist activity at 5-HT2B receptors. Thus, previous drugs with 5-HT2B receptor agonist activity have been found to have life threatening side effects such as cardiac valvulopathy (Rothman, R., Baumann, M., Savage, J., Rauser, L, McBride, A., Hufeisen, S., Roth, B. L. “Evidence for Possible Involvement of 5-HT2B Receptors in the Cardiac Valvulopathy Associated with Fenfluramine and other Serotonergic Medications” Circulation 2000, 102, 2836; Fitzgerald, L., Bum, T., Brown, B., Patterson, J., Corjay, M., Valentine, P., Sun, J-H., Link, J., Abbaszade, I., Hollis, J., Largent, B., Hartig, P., Hollis, G., Meunier, P., Robichaud, A, Robertson, D. “Possible Role of Valvular Serotonin 5-HT2B Receptors in the Cardiopathy Associated with Fenfluramine” Mol. Pharmacol. 2000, 57, 75) and pulmonary hypertension (Launay, J., Herve, P., Peoc'h, K., Toumois, C., Callebert, J., Nebigil, C., Etienne, N., Drouet, L., Humbert, M., Simonneau, G., Maroteaux, L. “Function of the Serotonin 5-Hydroxytryptamine 2B Receptor in Pulmonary Hypertension” Nature Med. 2002, 8, 1129). Furthermore, persons with pre-existing cardiovascular issues could be excluded from accessing the remarkable efficacy potential of psychedelic compounds and medicines, because they would be more susceptible to the undesirable cardiovascular changes that could be produced even by infrequent use of psychedelic molecules that activate the 5-HT2B receptor.

The development of 5-HT2C receptor agonists acting in the brain has been focused on treating obesity (Bickerdike, M., Vickers, S., Dourish, C. “5-HT2C Receptor Modulation and the Treatment of Obesity” Diabetes Obes. Metab. 1999, 1, 207; Martin, J., Bos, M., Jenck, F., Moreau, J-L., Mutel, V., Sleight, A., Wichmann, J., Andrews, J., Berendsen, H., Broekkamp, C., Ruight, G., Kohler, C., van Delft, A. M. L. “5-HT2C Receptor Agonists: Pharmacological Characteristics and Therapeutic Potential” J. Pharm. Exp Ther. 1998, 286, 913). Locaserin, a selective 5-HT2C agonist over 5-HT2B, was shown to have no increased risk of cardiac valvulopathy in patients and was FDA approved for treating obesity (Shukla A P, Kumar R B, Aronne L J (2015). “Lorcaserin HCl for the treatment of obesity” Expert Opinion on Pharmacotherapy. 76(16): 2531-8).

Sard et al. have described the synthesis and characterization of the 5-HT2 receptor activity of psilocin analogs (Sard H., Kumaran G., Morency C., Roth B. L., Toth B. A., He P., Shuster L. “SAR of psilocybin analogs: discovery of a selective 5-HT2C agonist” Bioorg. Med Chem. Lett. 2005, 75(20), 4555-9; Sard et al., Indole Compounds Useful as Serotonin Selective Agents; International Publication Number WO 2006/047302A1; Sard et al., Indole Compounds and Methods of Use Thereof, United States Patent Application Publication Number US2009/033822). The aim of these studies to identify 5-HT2C selective agonists, and psilocin analogs were identified with potent 5-HT2C agonist functional activity that were devoid of 5-HT2B agonist functional activity.

Selected analogs with this profile were also tested in an obsessive compulsive disorder (OCD) mouse behavior model versus psilocybin and psilocin as active reference compounds (Sard H., Kumaran G., Morency C., Roth B. L, Toth B. A., He P., Shuster L. “SAR of psilocybin analogs: discovery of a selective 5-HT2C agonist” Bioorg. Med. Chem. Lett. 2005, 15(20), 4555-9). In this report, the 1-methyl analog compound ofpsilocin, compound 1, was found to have high functional agonist selectivity for activation of phosphoinisitol hydrolysis on human 5-HT2C receptors vs the 5-HT2A and 5-HT2B receptors. For the human 5-HT2C receptor compound 1 was reported to have 12 nM potency with ca. 45% functional efficacy response vs serotonin (set at 100% a). At the human 5-HT2A receptor compound A was reported to have a 633 nM potency with ca. 30% functional efficacy response vs serotonin. Compound 1 was reported to be inactive as an agonist but a potent antagonist (38 nM) at 5-HT2B receptors. Finally, efficacy in a mouse OCD model demonstrated for compound 1 was ascribed to 5-HT2C receptor agonist activity. Compound 1, was later found to be efficacious in the mouse head twitch assay model for 5-HT2A activity but with much lower potency relative to psilocin (Halberstadt, A. L., Koedood, L. Powell, S. B. and Geyer, M. A. “Differential contributions of serotonin receptors to the behavioral effects of indoleamine hallucinogens in mice” Journal of Psychopharmacology, 2011, 25, 1548-1561). Compound 6, the 1-butyl analog of psilocin was reported to a selective for 5-HT2C agonist but with much lower potency (ca. 600 nM) indicating dramatic loss of potency with increasing size of the group substitution at the 1-position indole nitrogen.

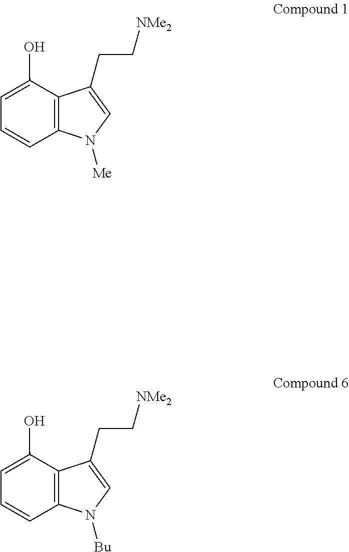

While psilocybin has recognized therapeutic potential for treating diverse CNS diseases and disorders including treatment-resistant depression (TRD), alcoholism, tobacco use, and cluster headache, there is an unmet need for safer drugs and analogs of psilocin that maintain 5-HT2A receptor agonist activity but that lack cardiotoxic 5-HT2B agonist activity.

Compounds with this selectivity profile for 5-HT2A over 5-HT2B are described as having broad potential to treat a variety of CNS diseases. However, because psilocybin does not fit this particular profile, its potential application as a drug therapy remains limited. It would be desirable to create tryptamine 5HT2A agonists that have some of the same pharmacological properties as psilocin but lack its 5-HT2B agonist activity, which has been linked to undesirable side effects such as cardiac valvulopathy.

### SUMMARY OF THE INVENTION

It is an object of the present disclosure to obviate or mitigate at least one of the above-mentioned disadvantages of the prior art.

It is another object of the present disclosure to provide a novel benzyl-tryptamine compound, preferably a novel N-methoxybenzyl-tryptamine compound.

It is another objection of the present disclosure to provide a novel process for producing the present benzyl-tryptamine compound, preferably the present N-methoxybenzyl-tryptamine compound.

Accordingly, in one of its aspects, the present disclosure provides a compound of Formula (I):

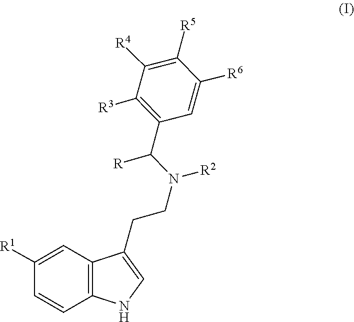

and any pharmaceutically acceptable salt or zwitterion thereof;
wherein:
R is hydrogen, methyl or ethyl;
R¹ is hydrogen or C₁-C₂ alkoxy;
R² is methyl or a C₂-C₄ group which may be saturated or unsaturated, branched or linear; and
R³, R⁴, R⁵ and R⁶ each are independently selected from hydrogen, hydroxyl, halogen, methyl optionally substituted with hydroxy, methoxy, ethoxy, and a saturated or unsaturated C₂-C₃ that may be optionally substituted with hydroxyl, with the provisos that: (i) at least two of R⁴, R⁵, R⁶ and R⁷ must be hydrogen, and (ii) R³, R⁴, R⁵ and R⁶ may be selected such that an adjacent pair thereof join to form a ring having at least 5 members.

In another of its aspects, the present disclosure provides a pharmaceutical composition comprising a compound of Formula (I):

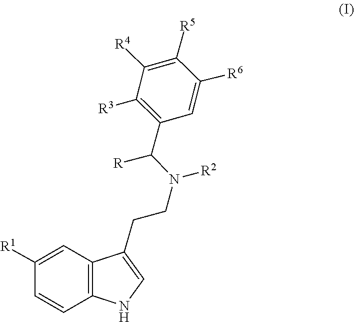

and any pharmaceutically acceptable salt or zwitterion thereof;
wherein:
R is hydrogen, methyl or ethyl;
R¹ is hydrogen or C₁-C₂ alkoxy;
R² is methyl or a C₂-C₄ group which may be saturated or unsaturated, branched or linear; and
R³, R⁴, R⁵ and R⁶ each are independently selected from hydrogen, hydroxyl, halogen, methyl optionally substituted with hydroxy, methoxy, ethoxy, and a saturated or unsaturated C₂-C₃ that may be optionally substituted with hydroxyl, with the provisos that: (i) at least two of R⁴, R⁵, R⁶ and R⁷ must be hydrogen, and (ii) R³, R⁴, R⁵ and R⁶ may be selected such that an adjacent pair thereof join to form a ring having at least 5 members.

The present inventor has unexpectedly discovered that the compounds of Formula (I) will bind the 5HT2B receptor but do not activate it and were shown to be an antagonist relative to serotonin at the 5-HT2B receptor and they are therefore potentially safe with respect to valvopathies, and yet the same compounds maintaining agonist activity at the 5-HT2A receptor and therefore are expected to have broad utility as a therapeutic agent. Thus, the compounds of Formula (I) appear to have a selectivity profile for 5-HT2A over 5-HT2B are believed to have a broad potential to treat a variety of CNS diseases while overcoming the above-described shortcomings of psilocybin. The present inventor believes that a significant advantage of the compounds of Formula (I) is that they will not behave as an agonist of 5-HT2B.

In a generally preferred embodiment, the compounds of Formula (I) have a 5-HT2A binding constant (Ki) determined according to the Cheng Prusoff equation of less than about 500 nM or less than about 300 nM or in the range of from about 0.1 nM to about 100 nm or in the range of from about 0.1 nM to about 30 nM or in the range of from about 0.1 nM to about 5 nM. Further, the compounds of the invention show selectivity that favors activation of 5-HT2A over 5-HT2B by a factor of 10 or more, 20 or more, 50 or more, 100 or more. In some embodiments, the molecules are potent activators of 5-HT2A but do not active the 5-HT2B, being antagonists or inactive yet still bind to the 5-HT2B receptor.

### BRIEF DESCRIPTION OF THE DRAWINGS

Embodiments of the present invention will be described with reference to the accompanying drawings, wherein like reference numerals denote like parts, and in which:

FIG. 1 illustrates the blood plasma profile associated with the results observed in Example 9.

### DETAILED DESCRIPTION OF THE PREFERRED EMBODIMENTS

As used herein, the term “about”, when used to describe a recited value, means within 5% of the recited value.

As used herein, the term “carrier” refers to a diluent, adjuvant, or excipient, with which a psilocybin analog described herein may be administered. Such pharmaceutical carriers can be liquids, such as water and oils, including those of petroleum, animal, vegetable or synthetic origin, such as peanut oil, soybean oil, mineral oil, sesame oil, and the like. The carriers can be saline, gum acacia, gelatin, starch paste, talc, keratin, colloidal silica, urea, and the like. In addition, auxiliary, stabilizing, thickening, lubricating, and coloring agents can be used. The pharmaceutically acceptable carriers are sterile.

As used herein, the term “chemical entity” refers to a compound having the indicated structure, whether in its “free” form (e.g., “free compound” or “free base” or “free acid” form, as applicable), or in a salt form, particularly a pharmaceutically acceptable salt form, and furthermore whether in solid state form or otherwise. In some embodiments, a solid state form is an amorphous (i.e., non-crystalline) form; in some embodiments, a solid state form is a crystalline form. In some embodiments, a crystalline form (e.g., a polymorph, pseudohydrate, or hydrate). Similarly, the term encompasses the compound whether provided in solid form or otherwise. Unless otherwise specified, all statements made herein regarding “compounds” apply to the associated chemical entities, as defined.

As used herein, the terms “comprising,” “having,” “including” and “containing,” and grammatical variations thereof, are inclusive or open-ended and do not exclude additional, un recited elements and/or method steps. The term “consisting essentially of” when used herein in connection with a composition, use or method, denotes that additional elements, method steps or both additional elements and method steps may be present, but that these additions do not materially affect the manner in which the recited composition, method or use functions. The term “consisting of” when used herein in connection with a composition, use or method, excludes the presence of additional elements and/or method steps.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19. Pharmaceutically acceptable salts of the compounds of this disclosure include those derived from suitable inorganic and organic acids and bases.

Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxyethanesulfonate, lactobionate, lactate, laurate, lauryl sulfate, malate, methanesulfonate, 2-naphthalenesulfonate, nicotinate, nitrate, oleate, palmitate, pamoate, pectinate, persulfate, 3-phenylpropionate, pivalate, propionate, stearate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, the term “subject” includes a mammal (e.g., a human, in some embodiments including prenatal human forms). In some embodiments, a subject is suffering from a relevant disease, disorder, or condition. In some embodiments, a subject is susceptible to a disease, disorder, or condition. In some embodiments, a subject displays one or more symptoms or characteristics of a disease, disorder, or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered. In some embodiments, a subject is a fetus, an infant, a child, a teenager, an adult, or a senior citizen (i.e., the subject is of advanced age, such as older than 50). In some embodiments, a child refers to a human between two and 18 years of age. In some embodiments, an adult refers to a human eighteen years of age or older.

### Chemical Entities of Formula (I)

The present disclosure provides a compound of Formula (I):

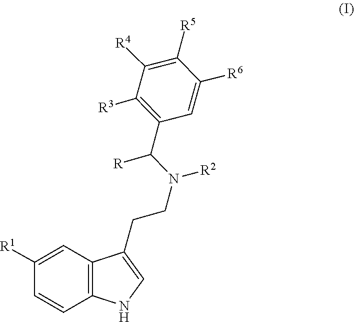

and any pharmaceutically acceptable salt or zwitterion thereof;
wherein:
R is hydrogen, methyl or ethyl;
R¹ is hydrogen or C₁-C₂ alkoxy;
R² is methyl or a C₂-C₄ group which may be saturated or unsaturated, branched or linear; and
R³, R⁴, R⁵ and R⁶ each are independently selected from hydrogen, hydroxyl, halogen, methyl optionally substituted with hydroxy, methoxy, ethoxy, and a saturated or unsaturated C₂-C₃ that may be optionally substituted with hydroxyl, with the provisos that: (i) at least two of R⁴, R⁵, R⁶ and R⁷ must be hydrogen, and (ii) R³, R⁴, R⁵ and R⁶ may be selected such that an adjacent pair thereof join to form a ring having at least 5 members.

In a preferred embodiment R² is selected from the group consisting of methyl, ethyl, n-propyl, i-propyl, 2-propenyl, 2-methyl-2-propenyl, 2-butenyl (trans) and 2-butenyl (cis).

In a preferred embodiment, R³, R⁴, R⁵ and R⁶ may be selected such that an adjacent pair thereof join to form a ring having at least 5 members, preferably from 5 to 8 members, preferably 5 or 6 members, preferably 5 members. In a preferred embodiment, the ring preferably contains at least 1, preferably 1 or 2 oxygen atoms.

In a preferred embodiment:
R¹ and R³ each are methoxy;
R² is methyl; and
R, R⁴, R⁵ and R⁶ each are hydrogen.

In a preferred embodiment:
R¹ and R³ each are methoxy;
R² is ethyl; and
R, R⁴, R⁵ and R⁶ each are hydrogen.

In a preferred embodiment:
R¹ and R³ each are methoxy;
R² is i-propyl; and
R, R⁴, R⁵ and R⁶ each are hydrogen.

In a preferred embodiment:
R¹ and R³ each are methoxy;
R² is 2-propenyl; and
R, R⁴, R⁵ and R⁶ each are hydrogen.

In a preferred embodiment:
R, R¹, R³, R¹ and R⁶ each are hydrogen;
R² is methyl; and
R⁴ is methoxy.

In a preferred embodiment:
R, R¹, R³, R⁴ and R⁶ each are hydrogen;
R² is methyl; and
R⁵ is methoxy.

In a preferred embodiment:
R, R¹, R⁵ and R⁶ each are hydrogen;
R² is methyl; and
R³ and R⁴ each are methoxy.

In a preferred embodiment:
R, R¹, R⁵ and R⁶ each are hydrogen;
R² is i-propyl; and
R³ and R⁴ each are methoxy.

In a preferred embodiment:
R, R¹, R³, R⁴, R⁵ and R⁶ each are hydrogen;
R² is methyl.

In a preferred embodiment:
R, R¹, R³, R⁵ and R⁶ each are hydrogen;
R² is 2-butenyl (cis); and
R⁴ is methoxy.

In a preferred embodiment:
R, R¹, R³, R⁵ and R⁶ each are hydrogen;
R² is 2-butenyl (trans); and
R⁴ is methoxy.

In a preferred embodiment:
R, R¹, R³, R⁵ and R⁶ each are hydrogen;
R² is 2-methyl-2-propenyl; and
R⁴ is methoxy.

In a preferred embodiment:
R, R¹, R³, R⁵ and R⁶ each are hydrogen;
R² is methyl; and
R⁴ is ethyl.

In a preferred embodiment:
R, R₁, R₃, R⁵ and R⁶ each are hydrogen;
R² is methyl; and
R⁴ is hydroxyl.

In a preferred embodiment:
R, R¹, R³, R⁵ and R⁶ each are hydrogen;
R² is methyl; and
R⁴ is bromine.

In a preferred embodiment:
R, R¹, R³, R⁵ and R⁶ each are hydrogen;
R² is methyl; and
R⁴ is hydroxyethyl.

In a preferred embodiment:
R, R¹, R³, R⁵ and R⁶ each are hydrogen;
R² is methyl; and
R⁴ is 2-propynyl.

In a preferred embodiment:
R, R¹, R⁵ and R⁶ each are hydrogen;
R² is methyl; and
R³ is methoxy, R⁴ is hydroxyl, and R³ and R⁴ join to form a 1,3-dioxolane group.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement hydrogen, carbon, nitrogen, oxygen, chlorine, or fluorine with ²H, ³H, ¹¹C, ¹³C, ¹⁴C, ¹³N, ¹⁵N, ¹⁷O, ¹⁸O, ³⁶Cl or ¹⁸F, respectively, are within the scope of this invention. Such compounds are useful, for example, as analytical tools, as probes in biological assays, or as therapeutic agents in accordance with the present disclosure. Additionally, incorporation of heavier isotopes such as deuterium (²H) can afford certain therapeutic advantages resulting from greater metabolic stability, for example, increase in vivo half-life, or reduced dosage requirements.

Unless otherwise stated, diastereomeric excess is expressed as % de, i.e., for diastereomers X and Y, the diastereomeric excess of X=((x−y)/(x+y))*100, where x and y are the fractions of X and Y, respectively.

Unless otherwise stated, enantiomeric excess is expressed as % ee, i.e., for enantiomers X and Y, the enantiomeric excess of X=((x−y)/(x+y))*100, where x and y are the fractions of X and Y, respectively.

### Formulations and Compositions

The present disclosure also provides pharmaceutically acceptable compositions which comprise a therapeutically effective amount of one or more of the compounds described herein, formulated together with one or more pharmaceutically acceptable carriers (additives) and/or diluents, and optionally, one or more additional therapeutic agents. While it is possible for a compound described herein to be administered alone, it is preferable to administer the compound as a pharmaceutical composition.

The term “pharmaceutical composition” means a composition comprising a compound of the present disclosure in combination with at least one additional pharmaceutically acceptable carrier. A “pharmaceutically acceptable carrier” refers to media generally accepted in the art for the delivery of biologically active agents to animals, in particular, mammals, including, i.e., adjuvant, excipient or vehicle, such as diluents, osmotic complement, preserving agents, fillers, flow regulating agents, disintegrating agents, wetting agents, emulsifying agents, suspending agents, sweetening agents, flavouring agents, perfuming agents, antibacterial agents, antifungal agents, lubricating agents, polymers, solubilizing agents, stabilizers, antioxidants and dispensing agents, depending on the nature of the mode of administration and dosage forms. Each carrier must be “acceptable” in the sense of being compatible with the other ingredients of the formulation and not injurious to the patient.

As used herein, “oral” administration includes swallowing for ingestion in the stomach or gut, and further includes lingual, sublingual, buccal and oropharyngeal administration. The compounds of the present disclosure can be administered for any of the uses or methods described herein by any suitable means, for example, orally, such as tablets, capsules (each of which may include sustained release or timed release formulations), pills, powders, granules, elixirs, suspensions (including nano suspensions, micro suspensions, spray-dried dispersions), syrups, and emulsions; sublingually (e.g. as thin films, effervescent tablets or tablets that dissolve spontaneously under the tongue); parenterally, such as by subcutaneous, intravenous, intramuscular injection, or infusion techniques (e.g., as sterile injectable aqueous or non-aqueous solutions or suspensions); nasally, including administration to the nasal membranes, such as by inhalation spray; or rectally such as in the form of suppositories.

The dosage regimen for the compounds described herein will, of course, vary depending upon known factors, such as the pharmacokinetic and pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient; and, the effect desired. The selected dosage level may also depend on the additional factors including the activity of the particular compounds and pharmaceutical compositions described herein, whether an ester, salt or amide substituent is of the compound is used, the time of administration, the rate of excretion or metabolism of the particular compound being employed, the rate and extent of absorption, the duration of the treatment, other drugs that may be administered to the patient, compounds and/or materials used in combination with the particular compound employed and like factors well known in the medical arts.

Generally, the dosage of the prodrug for a therapy session, when used for the indicated effects, will range between about 0.001 to about 500 mg per dose, preferably between about 0.01 to about 200 mg per dose, and most preferably between about 0.1 to about 50 mg per dose, such as 10, 20, 30, 40, 50, 100 or 200 mg. Intravenously, the most preferred doses will range from about 0.01 to about 10 mg/kg/minute during a constant rate infusion.

Compounds of this disclosure may be administered in a single daily dose, or the total daily dosage may be administered in multiple divided doses, such as two, three, or four times daily. Alternatively, the doses may be provided on a weekly, biweekly, or monthly basis. In a preferred embodiment, only one or two doses are required for an anti-depressant effect than may extend for 1, 2, 3 or 6 months, or more.

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl substituted hydroxypropyl cellulose, starch, pregelatinized starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray dried monohydrate, anhydrous and the like), mannitol, xylitol, dextrose, sucrose, sorbitol, microcrystalline cellulose, starch and dibasic calcium phosphate dihydrate.

Tablets may also optionally include surface active agents, such as sodium lauryl sulfate and polysorbate 80, and glidants such as silicon dioxide and talc. When present, surface active agents are typically in amounts of from 0.2 wt % to 5 wt % of the tablet, and glidants typically from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally are present in amounts from 0.25 wt % to 10 wt %, preferably from 0.5 wt % to 3 wt % of the tablet.

Other conventional ingredients include anti-oxidants, colorants, flavoring agents, preservatives and taste masking agents.

Exemplary tablets contain up to about 80 wt % drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet, dry, or melt granulated, melt congealed, or extruded before tableting. The final formulation may include one or more layers and may be coated or uncoated; or encapsulated.

The formulation of tablets is discussed in detail in “Pharmaceutical Dosage Forms: Tablets, Vol. 1”, by H. Lieberman and L. Lachman, Marcel Dekker, N.Y., N.Y., 1980 (ISBN 0 8247 6918 X).

A typical capsule for oral administration contains at least one of the compounds of the present disclosure (e.g., 25 mg), lactose (e.g., 75 mg), and magnesium stearate (e.g., 15 mg). The mixture is passed through a 60 mesh sieve and packed into a No. 1 gelatin capsule.

Liquid formulations include suspensions, solutions, syrups and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the present disclosure may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including micro needle) injectors, needle free injectors and infusion techniques.

Parenteral formulations are typically aqueous solutions which may contain excipients such as salts, carbohydrates and pH adjusting or buffering agents (preferably to a pH of from 3.0 and 7.0, preferably 4.0 to 6.0, and more preferably 4.5 to 5.5), but, for some applications, they may be more suitably formulated as a sterile non aqueous solution or as a dried form to be used in conjunction with a suitable vehicle such as sterile, pyrogen free water or pre-fabricated, ready-to-mix aqueous buffer. Osmotic agents may be included to control tonicity.

The preparation of parenteral kits for reconstitution at point-of-care under sterile conditions, for example, by lyophilization, may readily be accomplished using standard pharmaceutical techniques well known to those skilled in the art.

A typical injectable preparation is produced by aseptically placing at least one of the compounds of the present disclosure (e.g., 25 mg) into a vial as a sterile filtered solution, aseptically freeze-drying and sealing. For use, the contents of the vial are mixed with e.g. 2 mL of physiological saline for injection, optionally with an appropriate amount of osmotic complements and pH adjusters to achieve a slightly acidic to neutral pH (e.g., pH 4-7), to produce an injectable preparation with low irritation but retain solubility and/or stability of the prodrug.

Compounds of the present disclosure may be combined with soluble macromolecular entities, such as cyclodextrin and suitable derivatives thereof or polyethylene glycol containing polymers, in order to improve their solubility, dissolution rate, taste masking, bioavailability and/or stability for use in any of the aforementioned modes of administration.

Drug cyclodextrin complexes, for example, are found to be generally useful for most dosage forms and administration routes. Both inclusion and non inclusion complexes may be used. As an alternative to direct complexation with the drug, the cyclodextrin may be used as an auxiliary additive, i.e. as a carrier, diluent, or solubilizer. Most commonly used for these purposes are alpha, beta and gamma cyclodextrins, examples of which may be found in International Publication Numbers WO 91/11172, WO 94/02518 and WO 98/55148.

Regardless of the route of administration selected, the compounds of the present disclosure, which may be used in a suitable hydrated form, and/or the pharmaceutical compositions of the present disclosure, are formulated into pharmaceutically acceptable dosage forms by conventional methods known to those of skill in the art. Actual dosage levels of the active ingredients in the pharmaceutical compositions of this disclosure may be varied so as to obtain an amount of the active ingredient which is effective to achieve the desired therapeutic response for a particular patient, composition, and mode of administration.

A physician or veterinarian having ordinary skill in the art can readily determine and prescribe the effective amount of the pharmaceutical composition required. For example, the physician or veterinarian could start doses of the compounds of the present disclosure employed in the pharmaceutical composition at levels lower than that required in order to achieve the desired therapeutic effect and gradually increase the dosage until the desired effect is achieved.

In general, a suitable daily dose of a compound of the present disclosure will be that amount of the compound which is the lowest dose effective to produce a therapeutic effect. Such an effective dose will generally depend upon the factors described above.

As used herein, a “therapeutically effective amount” refers to that amount of a compound being administered which will relieve to some extent one or more of the symptoms of the disorder being treated. In reference to the treatment of depression, a therapeutically effective amount refers to that amount which has the effect of reducing the severity of depression. Depression severity may be assessed using well-known structured assessment tools such as Structured Clinical Interview for DSM-5 (SCID-5) and the GRID-Hamilton Depression Rating Scale (GRID-HAMD). A therapeutically effective amount may be less than that required for a psychedelic state.

An effective dosage can be administered in one or more administrations. For the purposes of this present disclosure, an effective dosage of drug, compound, or pharmaceutical composition is an amount sufficient to accomplish prophylactic or therapeutic treatment either directly or indirectly. As is understood in the clinical context, an effective dosage of drug, compound or pharmaceutical composition may or may not be achieved in conjunction with another therapy, drug, compound or pharmaceutical composition.

### Therapeutic Methods and Uses

Treatment with the novel compounds of the present disclosure may substantially alleviate clinical or subclinical depression and may avoid relapse, particularly if used in combination with psychotherapy for the treatment of depression. It is known that administration of an effective dose of psilocybin produced rapid and large reductions in depressive symptoms, and many subjects achieve remission through a four-week follow up (Davis et. al.). Without restriction to a theory, it is believed that the psychedelic state is associated with the beneficial effects, however, some compounds which are 5HT2A agonists may provide the desired therapeutic effect without the psychedelic state. One aspect of the present disclosure comprises prodrugs of those 5HT2A agonists which do provide a beneficial therapeutic state.

In general, the present disclosure includes the use of a compound of the present disclosure herein, to treat any disease or disorder which may be alleviated by a 5HT2A agonist, or the use of a compound of the present disclosure herein to manufacture a medicament to treat any disease or disorder which may be alleviated by a 5HT2A agonist, or a method of treating any disease or disorder which may be alleviated by a 5HT2A agonist.

In some embodiments, the invention may comprise the use of the compounds of the present disclosure to treat mental disorders. In some embodiments, the invention may comprise the use of the compounds of the present disclosure to treat depression, and particularly drug resistant depression. Other conditions that may be treated include: anxiety disorders, including anxiety in advanced stage illness (e.g., cancer) as well as generalized anxiety disorder, depression including major depressive disorder, postpartum depression, cluster headaches, obsessive compulsive disorder, personality disorders including conduct disorder, drug disorders including: alcohol dependence, nicotine dependence, opioid dependence, cocaine dependence and other addictions including gambling disorder, eating disorder and body dysmorphic disorder, chronic pain or chronic fatigue.

In some embodiments, the invention may comprise the use of the compounds of the present disclosure to treat metabolic syndrome and insulin resistance.

In some embodiments, the invention may comprise a method of treating mental disorders comprising administering to a subject in need thereof a therapeutically effective amount of a compound of the present disclosure. In one embodiment, there is provided a method of treating depression comprising administering to a subject in need thereof therapeutically effective amount of a compound of the present disclosure. The depression may be drug-resistant depression or major depressive disorder.

For example, a patient diagnosed with depression may be screened prior to treatment, and then prepared for a dosing session by a trained psychotherapist. Within a dosing session, a compound of the present disclosure may be administered by injection of a sterile solution at a rate of 0.01-0.3 mg/kg to the patient. The patient is preferably seated for the duration of the session while being blindfolded. For safety, a trained health care professional may monitor the patient throughout the dosing session, which may last up to 12 hours. In some cases, music may be played for the patient. When the health care professional can determine that the drug substance has cleared, the psychotherapist may assist the patient with any questions relating to the psychedelic experience, and then the patient may be discharged.

To further alleviate any anxiety that may occur relative to therapy, the physician may prefer to divide the therapeutic dose and thereby reduce the initial onset of psychoactivity before applying the full complement of the dosage to achieve the full effect.

In some embodiments, treatment with a compound of the present disclosure may be combined with concomitant treatment with another anti-depressant drugs, either concurrently or consecutively. In preferred embodiments, treatment with a compound of the present disclosure is combined with psychotherapy, which may be applied prior to or after treatment. If prior to, the session may focus the patient on the intent of treatment. If after, psychotherapy is preferably performed within 48 hours of the dosing session to help the patient integrate any feelings, emotions, visions or thoughts that may have occurred during the session, as well as to allow the psychotherapist may offer advice on how best to change thinking or behavior patterns so as to improve anti-depression outcomes. Psychotherapy may continue as needed after the dosing session, for example, up to an additional 3 months, to help the patient integrate any experiences or learnings that occurred to the patient during the dosing session.

### EXAMPLES

Aspects of the present disclosure may be described with reference to the following Examples. These Examples are provided for the purpose of illustration only and should not be used to construe or limit the scope of the invention. All terms, names, abbreviations or acronyms are those commonly understood by those skilled in the art. Compounds shown in their zwitterionic form may readily be visualized in their neutral form by one skilled in the art, and vice versa.

### Example 1—Synthesis of 2-(N-(2-methoxybenzyl)-N-Methyl)aminoethyl)-5-Methoxy-1H-indole

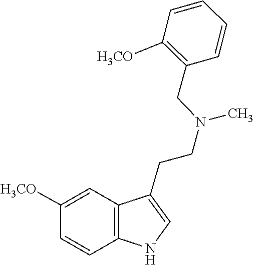 [SMILES: COc1ccc2[nH]cc(CCN(C)Cc3ccccc3OC)c2c1]

Step 1. To a 150 mL RBF with stir bar was added 5-methoxyindole-3-acetic (1.0 equiv), followed by anhydrous ACN (25 mL) under N₂. To this solution was then slowly added 2-methoxy-N-methylbenzylamine (2.0 equiv), then triethylamine (4.0 equiv). To the reaction mixture was then added n-propyl phosphonic acid cyclic anhydride (TP3) (8.68 g, 50% w/w in EtOAc). The reaction mixture was allowed to stir at room temperature overnight and monitored by TLC. The solvent was removed under vacuum. The residue was diluted with DCM (40 mL) and washed with brine (30 mL). The organic layer was separated, dried over Na₂SO₄, filtered and concentrated to dryness to give an orange oil (6.73 g of crude orange oil). Flash Chromatography (Biotage SNAP KP-SIL 340 g cartridge) using MeOH in EtOAc 0-5% gradient yielded 2.3 g of pure product as a yellow sticky solid (99% yield).

Step 2. To LiAlH₄ (5.0 equiv) in THF (100 ml) was added dropwise a solution containing 3.38 g product from step 1 in THF (60 mL) at 0° C. (ice bath). The reaction mixture was allowed to stir for 2 days as it slowly warmed to room temperature. The reaction was monitored by UPLC for disappearance of the starting material. After cooling the mixture to 0° C., the reaction was quenched by slow addition of MTBE (60 mL) followed by careful slow addition of 0.5 M NaOH (60 mL). MgSO₄ was added and the mixture was left stirring until a white precipitate was formed. The precipitate was filtered through a pad of Celite and washed with DCM. The filtrate was concentrated to dryness to give a greenish oil. Flash chromatography (Biotage SNAP KP-SIL 340 g cartridge) MeOH in EtOAc 0-5% in gradient yielded 3.78 g of crude orange oil (yield near quantitative). Purity UPLC=96%, QNMR (1,4-Dinitrobenzene; using peak at 6.7 (1H)) from compound in example)=96% (determined in two separate analyses). Exact mass by LCMS(MH+) 325.16. 1H NMR: 2.3 ppm (s, 3H, NMe), 2.6 ppm (m, 2H, —CH2N), 2.8 ppm (m, 2H, Indole-CH2-), 3.5 ppm (s, 2H, NCH2Ph), 3.7 ppm (s, 3H, OMe), 3.75 ppm (s, 3H, OMe), 6.7 ppm (m, 1H, aromCH), 6.9 ppm (m, 2H, aromCH), 6.95 ppm (d, 1H, aromCH), 7.1 ppm (s, 1H, aromCH), 7.2 ppm (m, 2H, aromCH), 7.4 ppm (m, 1H, aromCH), 10.7 ppm (m, 1H, NH).

Inhibition in functional GCPR assays for Adrenergic Alpha 1A and Alpha 2A were determined: IC50 (A1A): 1800 nM; IC50(A2A): 1700 nM.

### Example 2—5HT1A Competitive Binding Assay

Competitive binding assay (Eurofins Cerep) was performed using human recombinant 5-HT1A transfected to HEK-293 cells, [3H]8-OHDPAT (0.5 nM) and the test compound from Example 1 was tested at 8 concentrations ranging from 0.01 mM to 30 mM (Choi D S et al. FEBS Letters 1994, 352, 393). The analysis was performed using software developed at Cerep (Hill software) and validated by comparison with data generated by the commercial software SigmaPlot® 4.0 for Windows® (© 1997 by SPSS Inc). The binding constant (Ki 540 nM) were calculated using the Cheng Prusoff equation. The compound is a modest agonist at the 5HT1A receptor, but prefers the 5HT2A receptor (Ki 110 nM) by about 5-fold selectivity.

### Example 3—5HT2A Competitive Binding Assay

Competitive binding assay (Eurofins Cerep) was performed using human recombinant 5-HT2A transfected to HEK-293 cells, 1251-DOI (0.1 nM) and the test compound from Example 1 was tested at 8 concentrations ranging from 0.01 mM to 30 mM (Choi D S et al. FEBS Letters 1994, 352, 393). The analysis was performed using software developed at Cerep (Hill software) and validated by comparison with data generated by the commercial software SigmaPlot® 4.0 for Windows® (© 1997 by SPSS Inc). The binding constant (Ki 110 nM) were calculated using the Cheng Prusoff equation. The compound is approximately equipotent to psilocybin based on data from the http:/PDSP.unc.edu/databases/pdsp.php.

### Example 4—5HT2A Functional Assay

Competitive binding assay (Eurofins Cerep) was performed using human recombinant 5-HT2A transfected to HEK-293 cells, serotonin (30 nM) and the test compound from Example 1 was tested at 8 concentrations ranging from 0.01 mM to 30 mM (Choi D S et al. FEBS Letters 1994, 352, 393). The analysis was performed using software developed at Cerep (Hill software) and validated by comparison with data generated by the commercial software SigmaPlot® 4.0 for Windows® (© 1997 by SPSS Inc). The results show agonism at 5HT2A with EC50 of 520 nM reaching 80% maximum efficacy at the highest concentrations.

### Example 5—5HT2B Competitive Binding Assay

Competitive binding assay was performed (Eurofins Cerep) using human recombinant 5-HT1A transfected to CHO cells, 1251-DOJ (0.2 nM) and the test compound from Example 1 was tested at 8 concentrations ranging from 0.01 mM to 30 mM, (Choi D S et al. FEBS Letters 1994, 352, 393). The analysis was performed using software developed at Cerep (Hill software) and validated by comparison with data generated by the commercial software SigmaPlot® 4.0 for Windows® (© 1997 by SPSS Inc). No binding constant could be determined (Ki 180 nM) using the Cheng Prusoff equation. The compound will bind to the 5HT2B receptor with similar strength to the 5HT2A receptor (Ki 10 nM).

### Example 6—5HT2B (Isotol Phosphate, IP1) Functional Assay

A functional assay was performed (Eurofins Cerep) using human recombinant 5-HT2B transfected to CHO cells and the test compound from Example 1 was tested at 8 concentrations ranging from 0.01 mM to 30 mM, (see Porter, R H P et al. Brit. J. Pharmacol. 1999, 128, 13. Serotonin (1 uM) was used as a control. Quantification of myo-Inositol I phosphate was performed using HTRF. The analysis was performed using software developed at Cerep (Hill software) and validated by comparison with data generated by the commercial software SigmaPlot® 4.0 for Windows® (© 1997 by SPSS Inc). EC50 could not be determined for lack of activity of the compound at the receptor. Combined with the results of Example 4, this suggests that the compound will bind to the 5HT2B receptor, but does not generate any functional activity of the receptor and is thus acting as a neutral agonist or antagonist at therapeutic levels.

### Example 7—5HT2B Functional Antagonist Assay

A antagonist functional assay was performed (Eurofins Cerep) using human recombinant 5-HT2B transfected to CHO cells, with background serotonin (10 uM) and the test compound from Example 1 was tested at 8 concentrations ranging from 0.01 mM to 30 mM (see Porter, R H P et al. Brit. J. Pharmacol. 1999, 128, 13). Quantification of myo-Inositol-1-phosphate was performed using HTRF. The analysis was performed using software developed at Cerep (Hill software) and validated by comparison with data generated by the commercial software SigmaPlot® 4.0 for Windows (© 1997 by SPSS Inc). The results show that the Compound from Example 1 is a full antagonist with IC50<10 uM, when determined using the Cheng Prusoff equation.

### Example 8—5HT2C Competitive Binding Assay

Competitive binding assay was performed (Eurofins Cerep) using human recombinant 5-HT1A transfected to CHO cells, 1251-DOI (0.2 nM) and the test compound from Example 1 was tested at 8 concentrations ranging from 0.01 mM to 30 mM, (Choi D S et al. FEBS Letters 1994, 352, 393). The analysis was performed using software developed at Cerep (Hill software) and validated by comparison with data generated by the commercial software SigmaPlot® 4.0 for Windows® (© 1997 by SPSS Inc). No binding constant could be determined (Ki 680 nM) using the Cheng Prusoff equation. The compound is a modest agonist at the 5HT2C receptor but prefers the 5HT2A receptor (Ki 110 nM) by about 6-fold selectivity.

### Example 9—Pharmacokinetics after i.v. Administration in Rats

The compound of Example 1 was dispersed in water containing phosphate buffered saline at a rate of 1 mg/ml and then acidified to pH 4 to create a solution. The solution was administered to each of 3 rats (ca. 300 g each) at a rate of 1.0 mg/kg (ca. dose 0.33 ml, dose volume 0.33 mg) via a catheter placed in the jugular vein. Animals were observed over a period of 12 h with counting the cumulative number head-twitch actions over each 10 min up to 2 hours and then for 10 min each at 3, 3.5 and 4 hours. Blood samples (0.25 ml) were collected via the catheter using 1 ml syringes into 0.8 ml K2EDTA tubes at 0.0833, 0.25, 0.5, 0.75, 1, 2, 4, 6 hours after the dose and placed on wet ice until processing. Psychological saline (0.25 ml) was reinjected after each blood draw via the catheter to the animal to flush the catheter and replenish blood volumes. The blood samples collected were centrifuged (3200 g, 5 min, 4 C) within 5 min of collection and the plasma recovered and placed in a cryovial and frozen in liquid nitrogen and stored at −80 C thereafter until analysis. A bioanalytical method was developed to quantify the compound in plasma using a Sciex 6500 Q-trap MSMS equipped with a standard LC system and after calibration with the compound of the example.

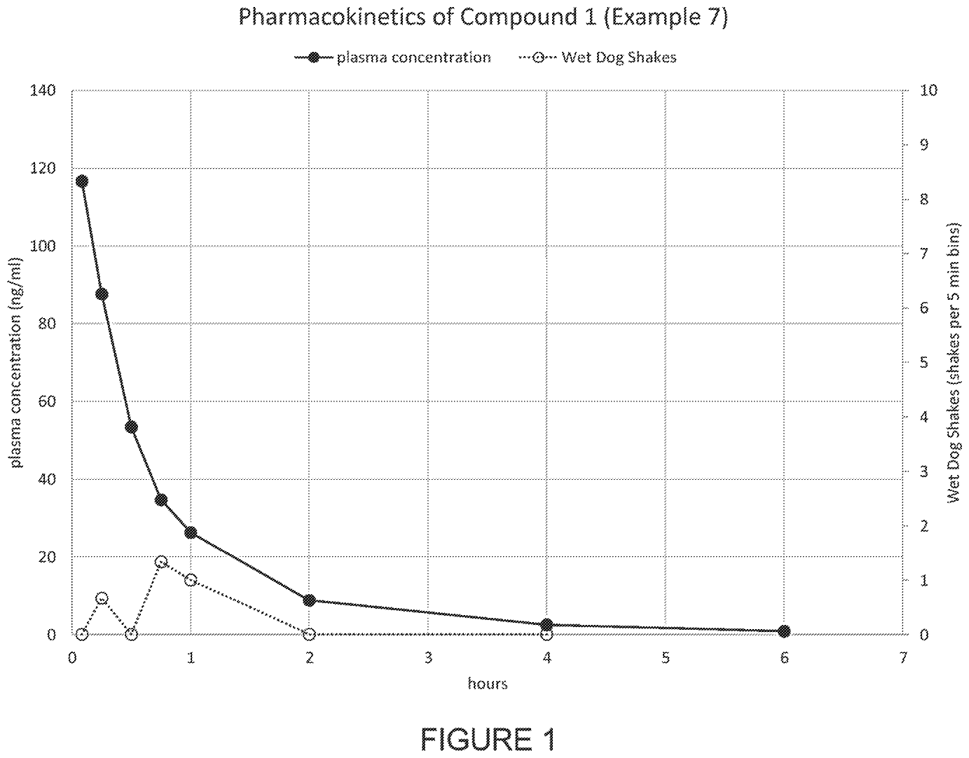

The blood plasma profile is shown in the FIG. 1. The mean plasma half-life was 66 minutes. There was no significant head twitch activity during the duration of the rat i.v. PK experiment indicating reduced hallucinogenic potential of the compound, despite showing 5HT2A receptor binding and functional assays (Examples 3 and 4 above). A non-hallucinogenic 5HT2A agonist could have significant potential utility in treating mood disorders and overcome the requirement for monitoring requirements typical of similar 5HT2A agonists in the same class, which produce a hallucinogenic state.

### Example 10—Synthesis of 2-(N-(3-methoxybenzyl)-N-Methyl)aminoethyl)-1H-indole

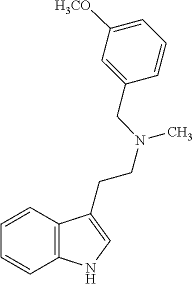 [SMILES: COc1cccc(CN(C)CCc2c[nH]c3ccccc23)c1]

The 2-step synthesis process, amide coupling, followed by reduction, in example 1 was performed using 3-methoxy-N-methylbenzylamine (2.0 equiv) instead of the 2-methoxy-modified amine described in Example 1. Yield of step 1: 85%; Yield of step 2: 20/a. Purity UPLC: 98%, QNMR (1,4-Dinitrobenzene; using peak at 6.7 (1H)) from compound in example): 96%. Exact mass by MS (MH+): 295.0. 1 H NMR (dmso-d6): 2.3 ppm (s, 3H, NMe), 2.6 ppm (m, 2H, —CH2N), 2.8 ppm (m, 2H, Indole-CH2-), 3.5 ppm (s, 2H, NCH2Ph), 3.7 ppm (s, 3H, OMe), 3.75 ppm (s, 3H, OMe), 6.8 ppm (m, 1H, aromCH), 6.9 ppm (m, 2H, aromCH), 6.95 ppm (m, 1H, aromCH), 7.05 ppm (s, 1H, aromCH), 7.13 ppm (m, 2H, aromCH), 7.2 (d, 1H, aromCH), 7.4 ppm (m, 1H, aromCH), 10.7 ppm (m, 1H, NH). 5HT1A Binding Ki 510 nM. 5HT2A Ki 14 nM. 5HT2B Binding Ki 380 nM. 5HT2B functional agonist mode IC50 1050 nM (Efficacy <20% Y up to 30 uM). 5HT2C Binding Ki 42 nM.

### Examples 11-29

Below is provided details on the synthesis and testing of the following compounds. The results of testing of various of the following compounds is reported in Table 1 below (all units in Table 1 are nM).

| Example | Compound |
|---|---|
| 11 | 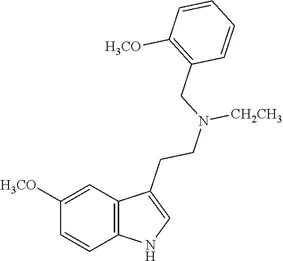 [SMILES: CCN(CCc1c[nH]c2ccc(OC)cc12)Cc1ccccc1OC] |
| 12 | 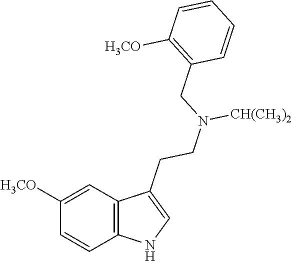 [SMILES: COc1ccc2[nH]cc(CCN(Cc3ccccc3OC)C(C)C)c2c1] |
| 13 | 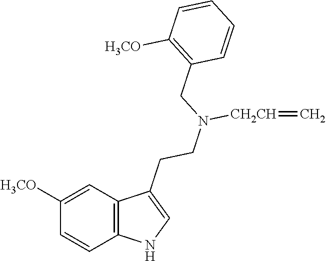 |
| 14 | 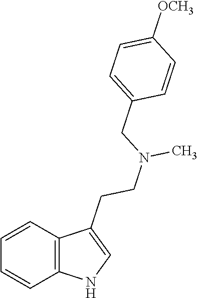 [SMILES: COc1ccc(CN(C)CCc2c[nH]c3ccccc23)cc1] |
| 15 | 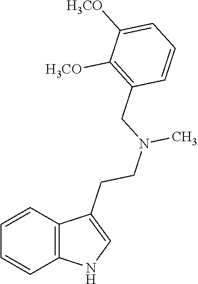 [SMILES: COc1cccc(CN(C)CCc2c[nH]c3ccccc23)c1OC] |
| 16 | 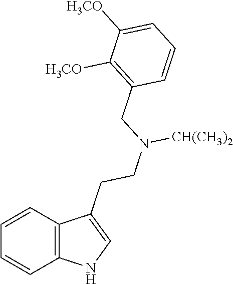 [SMILES: COc1cccc(CN(CCc2c[nH]c3ccccc23)C(C)C)c1OC] |
| 17 | 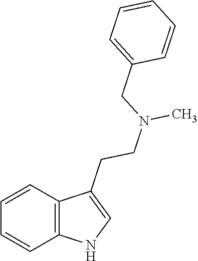 [SMILES: CN(CCc1c[nH]c2ccccc12)Cc1ccccc1] |
| 18 | 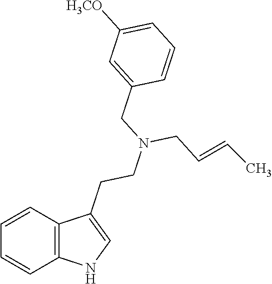 [SMILES: C/C=C/CN(CCc1c[nH]c2ccccc12)Cc1cccc(OC)c1] |
| 19 | 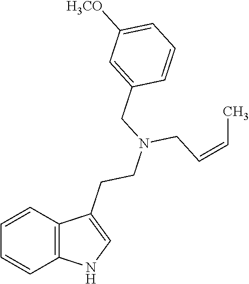 [SMILES: C/C=C\CN(CCc1c[nH]c2ccccc12)Cc1cccc(OC)c1] |
| 20 | 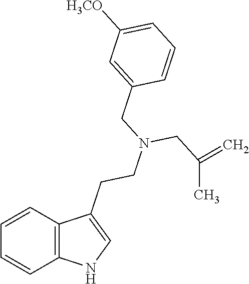 [SMILES: C=C(C)CN(CCc1c[nH]c2ccccc12)Cc1cccc(OC)c1] |
| 21 | 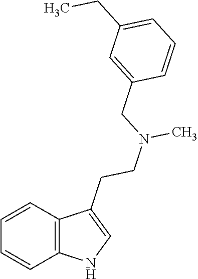 [SMILES: CCc1cccc(CN(C)CCc2c[nH]c3ccccc23)c1] |
| 22 | 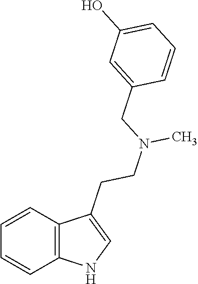 [SMILES: CN(CCc1c[nH]c2ccccc12)Cc1cccc(O)c1] |
| 23 | 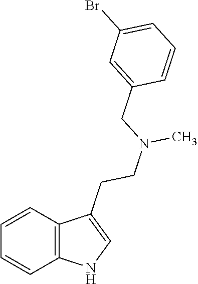 [SMILES: CN(CCc1c[nH]c2ccccc12)Cc1cccc(Br)c1] |
| 24 | 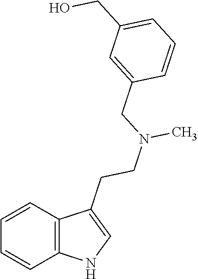 [SMILES: CN(CCc1c[nH]c2ccccc12)Cc1cccc(CO)c1] |
| 25 | 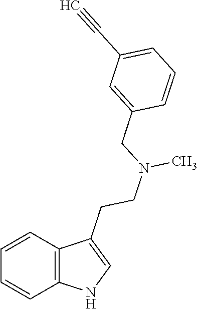 [SMILES: C#Cc1cccc(CN(C)CCc2c[nH]c3ccccc23)c1] |
| 26 | 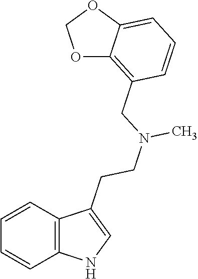 [SMILES: CN(CCc1c[nH]c2ccccc12)Cc1cccc2c1OCO2] |
| 27 | 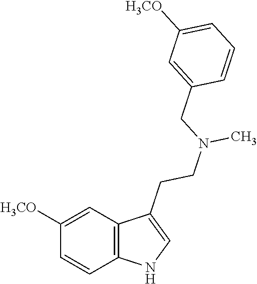 [SMILES: COc1cccc(CN(C)CCc2c[nH]c3ccc(OC)cc23)c1] |
| 28 | 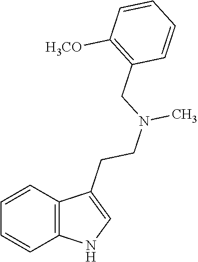 [SMILES: COc1ccccc1CN(C)CCc1c[nH]c2ccccc12] |
| 29 | 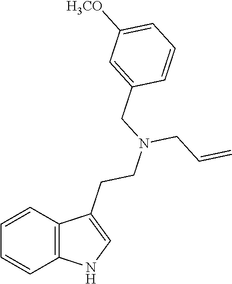 [SMILES: C=CCN(CCc1c[nH]c2ccccc12)Cc1cccc(OC)c1] |

### Example 11—Synthesis of N-ethyl-2-(5-methoxy-1H-indol-3-yl)-N-(2-methoxybenzyl)ethan-1-amine

The following reaction scheme was used:

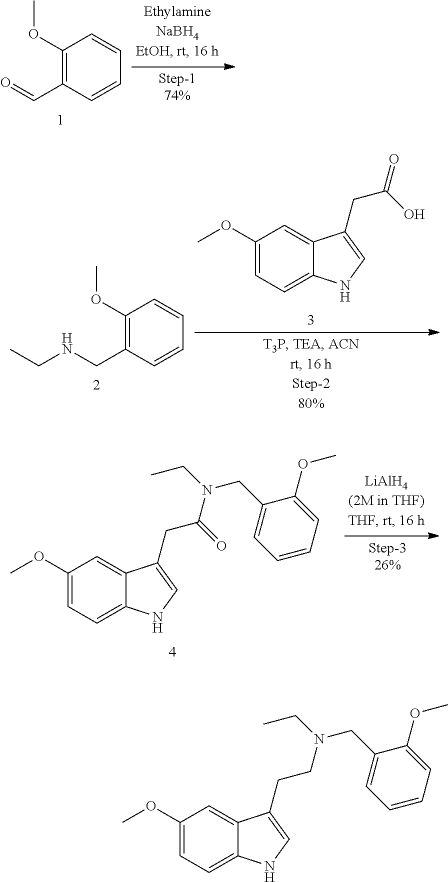

To a stirred solution of 2-methoxybenzaldehyde (3.0 g, 1.0 equiv) in EtOH (20 mL) was added Ethyl amine (0.99 g, 1.0 equiv) at room temperature. The reaction mixture was cooled to 0° C., stirred for 5 min., NaBH₄ (1.63 g, 2.0 equiv) was added portion wise. The reaction mixture was stirred at room temperature for 16 h. Progress of the reaction was monitored by TLC. After completion, the reaction mixture was concentrated in vacuo, diluted with water (50 mL), extracted with EtOAc (2×100 mL), dried over anhydrous Na₂SO₄, filtered and concentrated in vacuo to afford N-(2-methoxybenzyl) ethanamine (2, 2.7 g, 74%) as colorless thick syrup.

¹H NMR (400 MHz, DMSO-d₆) δ ppm 7.29 (dd, 0.1=7.25, 0.88 Hz, 1H, aromCH), 7.15-7.23 (m, 1H, aromCH), 6.95 (d, J=8.13 Hz, 1H, aromCH), 6.89 (t, J=7.38 Hz, 1H, aromCH), 3.77 (s, 3H, OMe), 3.66 (s, 2H, —CH2N), 2.53 (q, J=7.21 Hz, 2H, —CH2N), 1.02 (t, J=−7.13 Hz, 3H, —CH2Me).

To a stirred solution of 2-(5-methoxy-1H-indol-3-yl)acetic acid (0.62 g, 1.0 equiv) and N-(2-methoxybenzyl) ethanamine (0.5 g, 1.0 equiv) in CH₃CN (10 mL) was added TEA (1.8 mL, 12.12 mmol) at room temperature. The reaction mixture was cooled to 0° C., stirred for 5 min and 50% T₃P solution in EtOAc (3.9 mL, 2.0 equiv) was added dropwise. The reaction mixture was stirred at room temperature for 16 h. Progress of the reaction was monitored by TLC. After completion, the reaction mixture was concentrated in vacuo, diluted with water (30 mL), extracted with EtOAc (2×50 mL). Separated organic layer was dried over anhydrous Na₂SO₄, filtered and concentrated in vacuo. The crude obtained was purified by combi flash chromatography (20 to 30% EtOAc in heptane) to afford N-ethyl-2-(5-methoxy-1H-indol-3-yl)-N-(2-methoxybenzyl)acetamide (0.7 g, 65%) as colorless thick syrup. LCMS: Not done.

To a stirred solution of N-ethyl-2-(5-methoxy-1H-indol-3-yl)-N-(2-methoxybenzyl)-acetamide (0.7 g, 1.0 equiv) in THF (8 mL) was added a solution of 2M LiAlH₄ (2.0 equiv) in THF (2 mL) at 0° C. dropwise. The reaction mixture was stirred at room temperature for 16 h. Progress of the reaction was monitored by TLC. After completion, the reaction mixture was cooled to 0° C. and quenched with a saturated Na₂SO₄ solution (10 mL), white precipitate was filtered through pad of Celite and washed with EtOAc (100 mL). Filtrate was washed with water (30 mL), dried over anhydrous Na₂SO₄, filtered and concentrated in vacuo. The crude obtained was triturated with heptane (10 mL) to afford N-ethyl-2-(5-methoxy-1H-indol-3-yl)-N-(2-methoxybenzyl) ethan-1-amine (0.45 g, 67%) as an off white solid.

MS (EST) m/e [M+H]⁺: 339; HPLC purity: 99.84% (RT=5.9 min), ¹H NMR (400 MHz, DMSO-d₆) δ ppm 10.56 (s, 1H, indole-NH) 7.40 (dd, J=7.38, 1.38 Hz, 1H, AromCH) 7.15-7.21 (m, 2H, AromCH), 7.05 (d, J=2.13 Hz, 1H, AromCH), 6.81-6.98 (m, 3H, AromCH), 6.68 (dd, J=8.76, 2.38 Hz, 1H, AromCH), 3.77 (s, 3H, OMe), 3.70 (s, 3H, OMe), 3.63 (s, 2H, CH2), 2.77-2.84 (m, 2H, CH2), 2.65-2.71 (m, 2H, CH2), 2.59 (q, J=7.05 Hz, 2H, CH2), 1.04 (t, 1=7.07 Hz, 3H, —CH2Me).

### Example 12—Synthesis of N-(2-(5-methoxy-1H-indol-3-yl)ethyl)-N-(2-methoxybenzyl)propan-2-amine

The following reaction scheme was used:

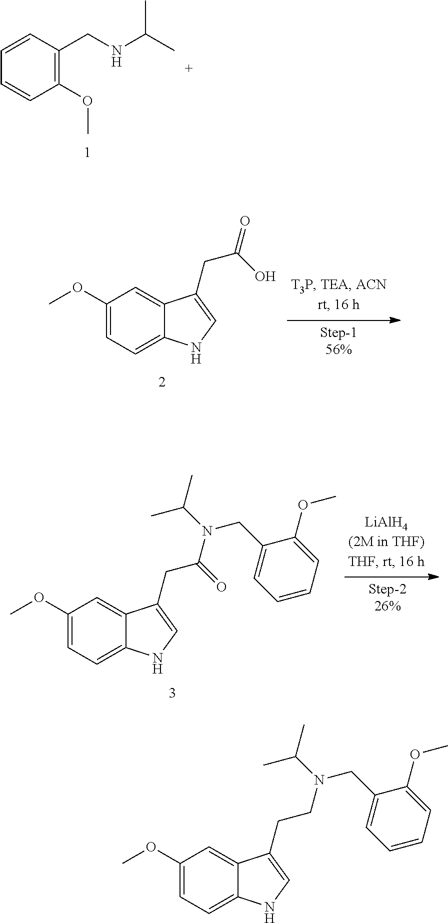

To a stirred solution of N-isopropyl-2-(5-methoxy-1H-indol-3-yl)-N-(2-methoxybenzyl) acetamide (1.0 g, 1.0 equiv) in THF (40 mL) was added dropwise a solution of 2M LiAlH₄ in THF (6.8 mL, 5.03 equiv) at 0° C. The reaction mixture was stirred at room temperature for 16 h. Progress of the reaction was monitored by TLC. After completion, the reaction mixture was cooled to 0° C. and quenched with a solution of saturated Na₂SO₄ in water (10 mL) white precipitation was filtered through pad of Celite, washed with EtOAc (100 mL). Filtrate was washed with water (50 mL), separated organic layer was dried over anhydrous Na₂SO₄, filtered and concentrated in vacuo. The crude obtained was purified by combi flash chromatography (1 to 5% MeOH in EtOAc) to afford N-(2-(5-methoxy-1H-indol-3-yl)ethyl)-N-(2-methoxybenzyl)propan-2-amine (0.34 g, 35%) as an colorless sticky oil.

MS (ESI) m/e [M+H]⁺: 353; HPLC purity: 97.03% (RT=1.8 min); ¹H NMR (400 MHz, DMSO-d₆) δ ppm 10.54 (s, 1H, indoleNH), 7.49 (dd, J=7.50, 1.50 Hz, 1H, aromCH), 7.14-7.22 (m, 2H, aromCH), 7.03 (d, J=2.25 Hz, 1H, aromCH), 6.87-6.96 (m, 2H, aromCH), 6.81 (d, J=2.38 Hz, 1H, aromCH), 6.67 (dd, J=8.69, 2.44 Hz, 1H, aromCH), 3.77 (s, 3H, OMe), 3.69 (s, 3H, OMe), 3.61 (s, 2H, CH2), 2.97-3.05 (m, 1H, CH), 2.62-2.76 (m, 4H, CH2), 1.01 (d, J=6.63 Hz, 6H, CH(CH₃)₂).

### Example 13—Synthesis of N-(2-(5-methoxy-1H-indol-3-yl)ethyl)-N-(2-methoxybenzyl)prop-2-en-1-amine

The following reaction scheme was used:

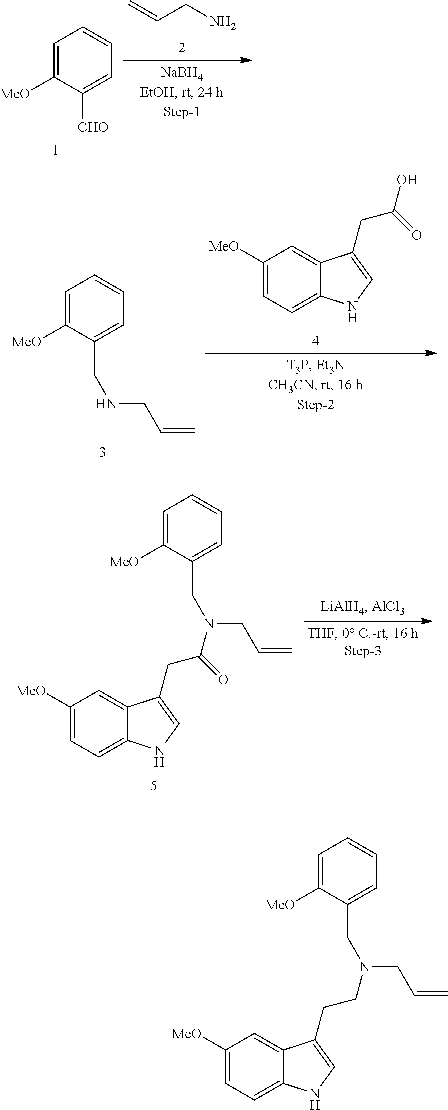

To a stirred solution of 2-methoxybenzaldehyde (1.0 g, 1.0 equiv) in EtOH (6 mL) was added prop-2-en-1-amine (0.46 g, 1.1 equiv) and stirred for at room temperature for 12 h. The reaction mixture was cooled to 0° C., stirred for 5 min., NaBH₄ (0.45 g, 1.6 equiv) was added portion wise. The reaction mixture was stirred at room temperature for 12 h. Progress of the reaction was monitored by TLC. After completion, the reaction mixture was quenched with water (10 mL) and the organic layer was concentrated in vacuo, diluted with water (50 mL) and extracted with EtOAc (2×100 mL). Separated organic layer was dried over anhydrous Na₂SO₄, filtered and concentrated in vacuo to N-(2-methoxybenzyl) prop-2-en-1-amine (0.7 g, 54%) as colorless thick syrup.

¹H NMR (400 MHz, DMSO-d₆) δ ppm 7.28-7.33 (m, 1H, aromCH) 7.18-7.27 (m, 1H, aromCH) 6.88-6.99 (m, 2H, aromCH) 5.87 (ddt, 0.1=16.93, 11.07, 5.32, 5.32 Hz, 1H, alkeneCH) 5.11-5.25 (m, 1H, alkeneCH) 5.07 (d, J=10.27 Hz, 1H, alkeneCH) 3.79 (s, 3H, OMe) 3.66 (s, 2H, NCH2) 3.17 (d, 0.1=4.89 Hz, 2H, NCH2)

To a stirred solution of N-(2-methoxybenzyl) prop-2-en-1-amine (0.7 g, 1.0 equiv) and 2-(5-methoxy-1H-indol-3-yl)acetic acid (0.81 g, 1.0 equiv) in ACN (10 mL) was added TEA (2.1 mL, 4.0 equiv) at room temperature. The reaction mixture was cooled to 0° C., stirred for 5 min and 50% T3P solution in EtOAc (5.0 mL, 2.0 equiv) was added dropwise. The reaction mixture was stirred at room temperature for 16 h. Progress of the reaction was monitored by TLC. After completion, the reaction mixture was concentrated in vacuo to afford crude, diluted with water (30 mL), extracted with EtOAc (2×50 mL). Separated organic layer was dried over anhydrous Na₂SO₄, filtered and concentrated in vacuo. The crude obtained was purified by combi flash chromatography (20 to 30% EtOAc in heptane) to afford N-allyl-2-(5-methoxy-1H-indol-3-yl)-N-(2-methoxybenzyl) acetamide (0.8 g, 55%) as colorless thick syrup. MS (ESI) m/e [M+H]: 365.

To a stirred solution of LAH (2M in THF, 7.7 mL, 3.9 equiv) in THF (50 mL) was added AlCl₃ (2.04 g, 4.0 equiv) portion wise at 0° C. The solution was stirred for 30 min at 0° C. N-allyl-2-(5-methoxy-1H-indol-3-yl)-N-(2-methoxybenzyl) acetamide (1.4 g, 1 equiv) in THF (25 mL) was added. The reaction mixture was stirred for 1 h at 0° C., then at room temperature for 12 h. Progress of the reaction was monitored by TLC. After was completion, the reaction mixture was cooled to 0° C. and quenched with 20% NaOH solution (10 mL) precipitate was filtered through pad of Celite, washed with EtOAc (100 mL). Filtrate was washed with water (50 mL), dried over anhydrous Na₂SO₄, filtered and concentrated in vacuo. The crude obtained was purified by combi flash (50 to 100% EtOAc in heptane) to afford N-ethyl-2-(5-methoxy-1H-indol-3-yl)-N-(2-methoxybenzyl) ethan-1-amine (0.8 g, 59%) as an off white solid.

MS (ESI) m/e [M+H]⁺: 351; HPLC purity: 99.43% (RT=6.06 min)¹H NMR (400 MHz, DMSO-d₆) δ ppm 10.55 (s, 1H, indoleNH), 7.40 (d, J=7.38 Hz, 1H, aromCH), 7.19 (d, J=8.76 Hz, 2H, aromCH), 7.00-7.06 (m, 1H, aromCH), 6.96 (d, J=8.13 Hz, 1H, aromCH), 6.90 (t, J=7.38 Hz, 1H, aromCH), 6.85 (d, J=2.13 Hz, 1H, aromCH), 6.67 (dd, J=8.69, 2.31 Hz, 1H, aromCH), 5.86-5.97 (m, 1H, alkeneCH), 5.24 (s, 1H, alkeneCH), 5.14 (d, J=10.26 Hz, 1H, alkeneCH), 3.77 (s, 3H, OMe), 3.70 (s, 3H, OMe) 3.65 (s, 2H, CH2), 3.19 (d, 0.1=6.13 Hz, 2H, CH2), 2.78-2.87 (m, 2H, CH2), 2.63-2.73 (m, 2H, CH2).

### Example 14—Synthesis of 2-(1H-indol-3-yl)-N-(4-methoxybenzyl)-N-methylethan-1-amine

The following reaction scheme was used:

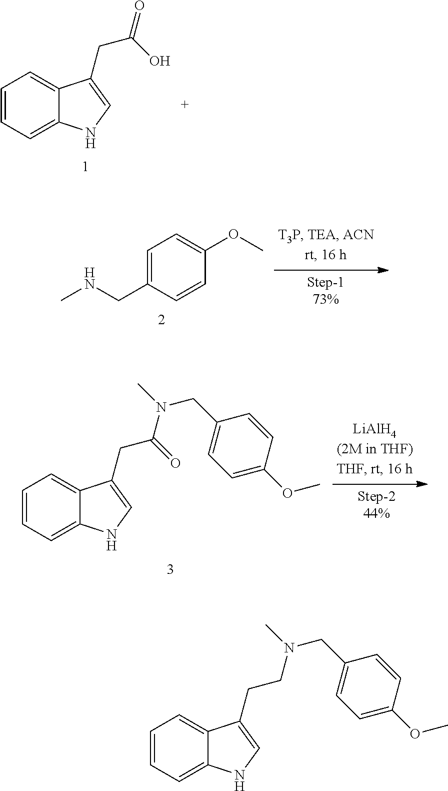

To a stirred solution of 2-(1H-indol-3-yl)acetic acid (0.85 g, 1.0 equiv) and 1-(4-methoxyphenyl)-N-methylmethanamine (0.88 g, 1.2 equiv) in CH₃CN (40 mL) was added TEA (2.58 mL, 4.04 equiv) at room temperature. The reaction mixture was cooled to 0° C., stirred for 5 min., 50% T₃P solution in EtOAc (6.1 mL, 2.02 equiv) was added dropwise. The reaction mixture was stirred at room temperature for 16 h. Progress of the reaction was monitored by TLC. After completion, the reaction mixture was concentrated in vacuo, diluted with water (30 mL), extracted with EtOAc (2×50 mL). Separated organic layer was dried over anhydrous Na₂SO₄, filtered and concentrated in vacuo. The crude obtained was purified by combi flash chromatography (20 to 30% EtOAc in heptane) to afford 2-(1H-indol-3-yl)-N-(4-methoxybenzyl)-N-methylacetamide (3, 1.1 g, 73%) as colorless oil.

MS (ESI) m/e [M+H]⁺: 308.9; ¹H NMR (400 MHz, CHLOROFORM-d) δ ppm 8.24 (s, 1H, indoleNH), 7.58-7.67 (m, 1H, aromCH), 7.35 (d, J=8.00 Hz, 1H, aromCH), 7.05-7.22 (m, 5H, aromCH), 6.82 (d, J=8.25 Hz, 2H, aromCH), 4.33-4.62 (m, 2H, CH2), 3.89 (s, 2H, CH2), 3.79 (s, 3H, OMe), 2.93 (s, 3H, NMe).

To a stirred solution of 2-(1H-indol-3-yl)-N-(4-methoxybenzyl)-N-methylacetamide (1.1 g, 1.0 equiv) in THF (40 mL) was added a solution of 2M LiAlH₄ in THF (4.46 mL, 2.5 equiv) at 0° C. The reaction mixture was stirred at room temperature for 16 h. Progress of the reaction was monitored by TLC. After completion, the reaction mixture was cooled to 0° C. and quenched with saturated Na₂SO₄ solution (10 mL) white precipitate was filtered through pad of Celite, washed with EtOAc (100 mL). Filtrate was washed with water (50 mL), dried over anhydrous Na₂SO₄, filtered and concentrated in vacuo. The crude obtained was triturated with n-heptane (10 mL) to afford 2-(1H-indol-3-yl)-N-(4-methoxybenzyl)-N-methylethan-1-amine (FF165, 0.47 g, 44%) as an off white solid.

MS (ESI) m/e [M+H]⁺: 295; HPLC purity: 98.04% (RT=5.2 min); ¹H NMR (400 MHz, DMSO-d₆) δ ppm 10.73 (s, 1H, indoleNH), 7.42 (d, J=7.8 Hz, 1H, aromCH), 7.31 (d, J=8 Hz, 1H, aromCH), 7.28-7.17 (m, 2H, aromCH), 7.11 (s, 1H), 7.04 (t, J=7.6 Hz, 1H, aromCH), 6.93 (t, J=7.6 Hz, 1H, aromCH), 6.86 (d, J=7.8 Hz, 2H, aromCH), 3.73 (s, 3H, OMe), 3.48 (s, 2H, CH2), 2.94-2.77 (m, 2H, CH2), 2.67-2.55 (m, 2H, CH2), 2.21 (s, 3H, NMe).

### Example 15—Synthesis of N-(2,3-dimethoxybenzyl)-2-(H-indol-3-yl)-N-methylethan-1-amine

The following reaction scheme was used:

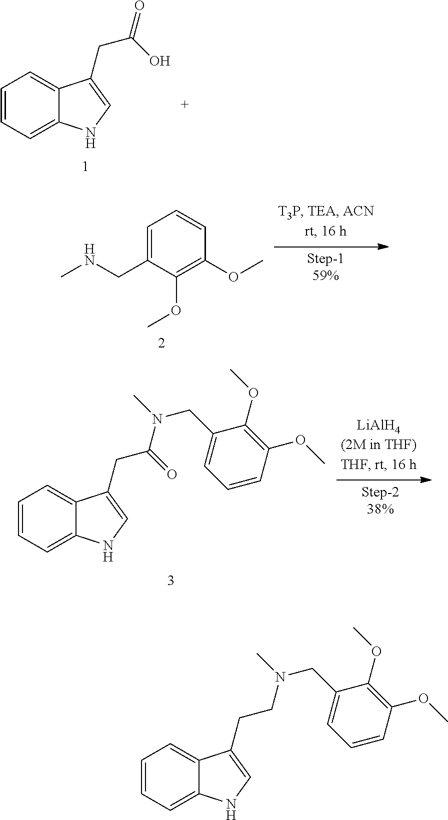

To a stirred solution of 2-(1H-indol-3-yl)acetic acid (0.87 g, 1.0 equiv) and Comp-2 (0.9 g, 4.97 mmol) in CH₃CN (10 mL) was added TEA (2.9 mL, 4.0 equiv) at room temperature. The reaction mixture was cooled to 0° C., stirred for 5 min and 50% T₃P solution in EtOAc (3.16 mL, 2.0 equiv) was added dropwise. The reaction mixture was stirred at room temperature for 16 h. Progress of the reaction was monitored by TLC. After completion, the reaction mixture was concentrated in vacuo and diluted with water (50 mL), extracted with EtOAc (2×100 mL). Separated organic layer was dried over anhydrous Na₂SO₄, filtered and concentrated in vacuo to afford crude. The crude obtained was purified by combi flash chromatography (20 to 30% EtOAc in heptane) to afford N-(2,3-dimethoxybenzyl)-2-(1H-indol-3-yl)-N-methylacetamide (3, 1.0 g, 59%) as colorless oil.

MS (ESI) m/e [M+H]⁺: 339; ¹H NMR (400 MHz, DMSO-d₆) δ ppm 10.88 (d, J=9.78 Hz, 1H, indoleNH), 7.53-7.61 (m, 1H, aromCH), 7.49 (d, J=7.34 Hz, 1H, aromCH), 7.27-7.40 (m, 1H, aromCH), 7.19 (d, J=13.20 Hz, 1H, aromCH), 6.87-7.10 (m, 3H, aromCH), 6.54-6.67 (m, 1H, aromCH), 4.45-4.65 (m, 2H, CH2), 3.76-3.85 (m, 6H, OMe), 3.70 (d, J=15.16 Hz, 3H, NMe), 2.96 (s, 2H, CH2).

To a stirred solution of N-(2,3-dimethoxybenzyl)-2-(1H-indol-3-yl)-N-methylacetamide (1.5 g, 1.0 equiv) in THF (50 mL) was added a solution of 2M LiAlH₄ in THF (4.88 mL, 2.02 equiv) at 0° C. dropwise. The reaction mixture was stirred at room temperature for 16 h. Progress of the reaction was monitored by TLC. After completion, the reaction mixture was cooled to 0° C. and quenched with saturated Na₂SO₄ solution (10 mL) white precipitate was filtered through pad of Celite, washed with EtOAc (70 mL). Filtrate was washed with water (50 mL), dried over anhydrous Na₂SO₄, filtered and concentrated in vacuo. The crude obtained was purified by combi flash chromatography (40 to 60% EtOAc in heptane) to afford N-(2,3-dimethoxybenzyl)-2-(1H-indol-3-yl)-N-methylethan-1-amine (0.37 g, 38%) colorless sticky oil.

MS (ESI) m/e [M+H]⁺: 325; HPLC purity: 97.62% (RT=5.9 min); ¹H NMR (400 MHz, DMSO-d₆) δ ppm 10.73 (s, 1H, indoleNH), 7.43 (d, J=7.88 Hz, 1H, aromCH), 7.31 (d, J=8.13 Hz, 1H, aromCH), 7.09-7.13 (m, 1H, aromCH), 6.97-7.06 (m, 2H, aromCH), 6.90-6.97 (m, 3H, aromCH), 3.78 (s, 3H, OMe), 3.68 (s, 3H, OMe), 3.53 (s, 2H, CH2), 2.81-2.93 (m, 2H, CH2), 2.59-2.70 (m, 2H, CH2), 2.24 (s, 3H, NMe).

### Example 16—Synthesis of N-(2-(1H-indol-3-yl)ethyl)-N-(2,3-dimethoxybenzyl)prop-2-an-1-amine

The following reaction scheme was used:

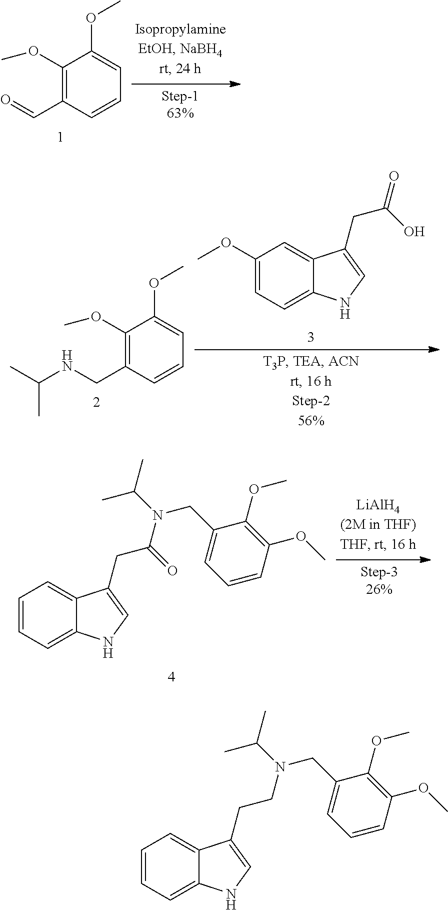

To a stirred solution of 2,3-dimethoxybenzaldehyde (3.0 g, 1.0 equiv) in EtOH (90 mL) was added Isopropyl ethyl amine (1.5 g, 1.5 equiv). The reaction mixture was stirred for 12 h at room temperature, cooled to 0° C. and NaBH₄ (1.33 g, 2.0 equiv) was added portion wise. The reaction mixture was stirred at room temperature for 12 h. Progress of the reaction was monitored by TLC. After completion, the reaction mixture was quenched with water (10 mL), the organic layer was concentrated in vacuo, diluted with water (50 mL), extracted with EtOAc (2×100 mL). Separated organic layer was dried over anhydrous Na₂SO₄, filtered and concentrated in vacuo to afford N-(2,3-dimethoxybenzyl)propan-2-amine (2.4 g, 63%) as colorless oil.

To a stirred solution of N-(2,3-dimethoxybenzyl)propan-2-amine (2.4 g, 1.0 equiv) and Comp-3 (2.0 g, 1.0 equiv) in CH₃CN (30 mL) was added TEA (6.1 mL, 4.0 equiv) at room temperature. The reaction mixture was cooled to 0° C., stirred for 5 min and 50% T₃P solution in EtOAc (14.6 mL, 2.0 equiv) was added dropwise. The reaction mixture was stirred at room temperature for 16 h. Progress of the reaction was monitored by TLC. After completion, the reaction mixture was concentrated in vacuo to afford crude, diluted with water (50 mL), extracted with EtOAc (2×100 mL). Separated organic layer was dried over anhydrous Na₂SO₄, filtered and concentrated in vacuo to afford crude. The crude obtained was purified by combi flash chromatography (20 to 30% EtOAc in heptane) to N-(2,3-dimethoxybenzyl)-2-(1H-indol-3-yl)-N-isopropylacetamide (2.5 g, 59%) as colorless sticky oil.

To a stirred solution of N-(2,3-dimethoxybenzyl)-2-(1H-indol-3-yl)-N-isopropylacetamide (1.5 g, 1.0 equiv) in THF (50 mL) was added dropwise a solution of 2M LiAlH₄ in THF (10.2 mL, 5.1 equiv) at 0° C. The reaction mixture was stirred at room temperature for 16 h. Progress of the reaction was monitored by TLC. After completion, the reaction mixture was cooled to 0° C. and quenched with saturated Na₂SO₄ solution (10 mL) white precipitation was formed and filtered through pad of Celite, cake was washed with EtOAc (100 mL). Filtrate was washed with water (50 mL), separated organic layer was dried over anhydrous Na₂SO₄, filtered and concentrated in vacuo to afford crude. The crude obtained was purified by Prep HPLC which was triturated with MeOH (5 mL) to afford N-(2-(1H-indol-3-yl)ethyl)-N-(2,3-dimethoxybenzyl)propan-2-amine (0.4 g, 28%) as off white solid.

MS (ESI) m/e [M+H]⁺: 353; HPLC purity: 98.36% (RT=8.23 min); ¹H NMR (400 MHz, DMSO-d₆) δ ppm 10.70 (s, 1H, indoleNH), 7.21-7.38 (m, 2H, aromCH), 6.93-7.11 (m, 4H, aromCH), 6.87-6.93 (m, 2H, aromCH), 3.77 (s, 3H, OMe), 3.69 (s, 3H, OMe), 3.61 (s, 2H, CH2), 2.90-3.04 (m, 1H, NCH), 2.68-2.77 (m, 2H, CH2), 2.60-2.67 (m, 2H, CH2), 0.99 (d, f=6.85 Hz, 6H, NCHMe).

### Example 17—Synthesis of N-(2-(1H-indol-3-yl)ethyl)-N-(benzyl)methan-1-amine

The following reaction scheme was used:

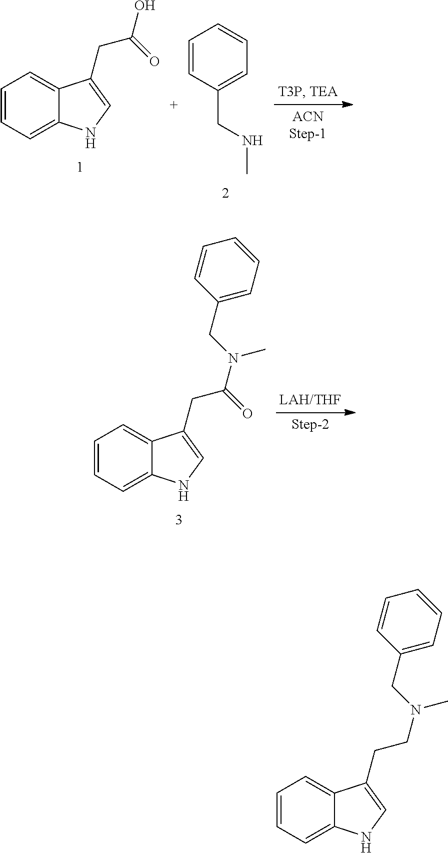

To a stirred solution of 2-(1H-indol-3-yl)acetic acid (1 g, 1.0 equiv) in 10 ml of ACN were added N-methyl-1-phenylmethanamine (0.8 g, 1.2 equiv) followed by T₃P (3.6 g, 2.0 equiv) and Et₃N (1.7 g, 3.0 equiv) at room temperature. The reaction mixture was stirred at RT for 16 h. Progress of the reaction was monitored by TLC. After completion, the reaction mixture was concentrated in vacuo, diluted with water (30 mL), extracted with EtOAc (2×50 mL). Separated organic layer was dried over anhydrous Na2SO4, filtered and concentrated in vacuo. The crude obtained was purified by combi flash chromatography to get the desired product as white colour solid (1.2 g, 75% yield).

To a stirred solution of N-benzyl-2-(1H-indol-3-yl)-N-methylacetamide (1.2 g, 1.0 equiv) in 3 ml of THF was added a solution of 2M LAH (0.3 g, 2.0 equiv) dropwise. The reaction mixture was stirred at 0° C. for 4 h under N₂ atmosphere. Progress of the reaction was monitored by TLC. After completion, the reaction mixture was quenched with a saturated Na₂SO₄ solution (10 mL), white precipitate was filtered through pad of Celite and washed with EtOAc (100 mL). Filtrate was washed with water (30 mL), dried over anhydrous Na₂SO₄, filtered and concentrated in vacuo. The crude obtained was purified by combi flash chromatography to afford N-benzyl-2-(1H-indol-3-yl)-N-methylethan-1-amine (FT232, 0.6 g, 52%) as a brown solid.

MS (ESI) m/e [M+H]⁺: 265; HPLC purity: 99.6% (RT=5.7 min), ¹H NMR (400 MHz, DMSO-d₆) δ=10.74 (s, 1H, indoleNH), 7.42 (d, 0.1=7.75 Hz, 1H, aromCH), 7.39-7.26 (m, 5H, aromCH), 7.25 (s, 1H, aromCH), 7.12 (d, J=2.0 Hz, 1H, aromCH), 7.04 (dt, J=2.0 Hz, 8.0 Hz, 1H, aromCH), 6.93 (dt, J=2.0 Hz, 8.0 Hz, 1H, aromCH), 3.56 (s, 2H, CH2), 2.88 (t, J=4.0 Hz, 2H, CH2), 2.64 (s, 2H, CH2), 2.24 (s, 3H, NMe).

### Example 18—Synthesis of N-(2-(1H-indol-3-yl)ethyl)-N-(3-ethylbenzyl)prop-2-en-1-amine

3-(aminoethyl)-1H-indole (1 equiv) was reacted with 3-methyoxybenzoic anhydride (1.2 equiv) in ethanol at 65 C for 7 h to fomr an intermediate imine which was then cooled to room temperature and reacted directly by the addition of NaBH4 (2 equiv). After quenching, extraction and evaporation of solvents, yielded the desired 3-(3-methoxybenzylaminoethyl)-1H-indole (1.1 g, 62%). This (0.6 g, 1 equiv) was then reacted with trans-1-bromo-2-butene (1.1 equiv) in a minimal amount of DMF using K2CO3 (2 equiv) at RT over 2 h. The product was isolated after extraction followed by flash chromatography to yield a semisolid (0.48 g, 57%). Purity (UPLC): 97.4%. MS MH+335. 1H-NMR (dmso-d6): 1.7 (d, 3H), 2.7 (m, 2H), 2.84 (m, 2H), 3.2 (m, 2H), 3.6 (m, 2H), 3.73 (s, 3H), 5.6 (m-m, 2H), 6.8 (dd, 1H), 6.9 (m, 3H), 7.05 (t, 1H), 7.08 (m, 1H), 7.2 (t, 1H), 7.3 (d, 1H), 7.4 (d, 1H), 10.7 (br s, 1H).

### Example 19—Synthesis of (Z)—N-(2-(1H-indol-3-yl)ethyl)-N-(3-methoxybenzyl)but-2-en-1-amine

The following reaction scheme was used:

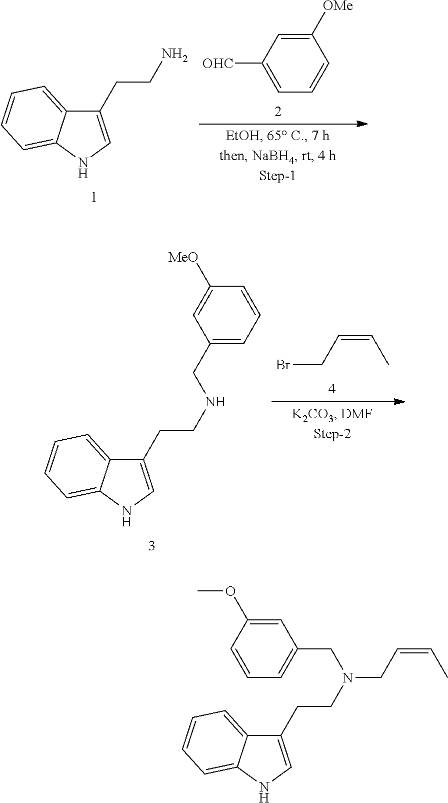

To a stirred solution of 2-(1H-indol-3-yl)ethan-1-amine (1.5 g, 1.0 equiv) in 40 mL of ethanol was added 3-methoxybenzaldehyde (1.5 g, 1.2 equiv) at room temperature. The reaction mixture was stirred at room temperature for 16 h. Then the reaction mixture was cooled to 0° C. and NaBH₄ (0.7 g, 2.0 equiv) was added portion wise and stirred further for 4 h. The progress of reaction was monitored by TLC. After completion, the reaction mixture was quenched with ice-water and extracted with ethyl acetate (50 ml×2). The organic layer was washed with brine solution and dried over anhydrous Na₂SO₄, and filtered, concentrated in vacuo. The crude was purified by Flash chromatography (Combi-Flash Column, 24 g redisep cartridge) using 100% DCM to 2% MeOH in DCM to get desired product as thick orange syrup (1.8 g. 69% yield).

To a solution of but-2-yn-1-ol (2.0 g, 1.0 equiv) in 50 mL of methanol was added Lindlar catalyst (0.2 g, 0.04 equiv) at room temperature under 50 psi of hydrogen atmosphere. The reaction mixture was stirred at room temperature for 3 h. The progress of reaction was monitored by TLC. After completion, the Reaction mixture was filtered through a pad of Celite. The filtrate was concentrated in vacuo gave crude compound ((Z)-but-2-en-1-ol) (1.8 g, 67% yield). The crude compound was used as such for next step.

To a stirred solution of (Z)-but-2-en-1-ol (1.8 g, 1.0 equiv) in 40 ml of diethyl ether at 0° C. was added PBr₃ (0.9 ml, 0.4 equiv) dropwise. The RM was stirred at room temperature for 3 h. The progress of reaction was monitored by TLC. After completion, the reaction mixture was quenched with ice-water and aqueous layer was extracted with diethyl ether (50 ml×2). Organic layer was washed with brine solution and dried over anhydrous Na₂SO₄, and filtered, concentrated in vacuo, to obtained yellow liquid, which was used as such for next step (0.6 g, 16% yield).

To a stirred solution of 2-(1H-indol-3-yl)-N-(3-methoxybenzyl)ethan-1-amine (1.0 g, 1.0 equiv) in 8 ml of DMF were added K₂CO₃ and (Z)-1-bromobut-2-ene (0.5 g, 1.1 equiv) dropwise. The RM was stirred at rt for 3 h. Completion of the reaction was checked by TLC. The RM was diluted with water (30 ml) and DCM (50 ml). The organic layer was separated and washed with water (30 ml×2). Then organic layer was dried over anhydrous Na₂SO₄, and filtered, concentrated in vacuo. The crude was purified by Flash chromatography (Combi-Flash Column, 24 g redisep cartridge) using 100% DCM to 2% MeOH in DCM to get desired product as thick orange syrup (0.5 g, 37% yield).

MS (EST) m/e [M+H]⁺: 335; HPLC purity: 98% (RT=6.2 min), ¹H NMR (400 MHz, DMSO-d₆) δ=10.74 (s, 1H, indoleNH), 7.39-7.29 (m, 2H, aromCH), 7.23 (s, 1H, aromCH), 7.12-6.99 (m, 2H, aromCH), 6.93 (d, J=6.8 Hz, 3H, aromCH), 6.81 (s, 1H, aromCH), 5.82-5.46 (m, 2H, alkeneCH), 3.72 (s, 3H, OMe), 3.62 (s, 2H, CH2), 3.11 (s, 2H, CH2), 2.86 (s, 2H, CH2), 2.70 (s, 2H, CH2), 1.73-1.55 (m, 3H, alkeneMe).

### Example 20—Synthesis of N-(2-(1H-indol-3-yl)ethyl)-N-(3-methoxybenzyl)2-methylprop-2-en-1-amine

3-(3-methoxybenzylaminoethyl)-1H-indole of Example 18 (0.61 equiv, 1 equiv) was alkylated with 3-Bromo-2-methylpropene (1.1 equivalent) in a manner identical to the method described in Example 18 to yield the desired compound (0.5 g, 60%). Purity (UPLC) 99.8%. MS MH+335. 1H-NMR (dmso-d6): 1.7 (s, 1H), 2.6 (m, 2H), 2.88 (m, 2H), 3 (s, 2H), 3.6 (s, 2H), 3.7 (s, 3H), 4.85 (s, 1H), 4.95 (s, 1H), 6.8 (d, 1H), 6.9 (mm, 3H), 7.1 (mm, 2H), 7.2 (t, 1H), 7.3 (d, 1H), 7.35 (d, 1H), 10.7 (br s, 1H).

### Example 21—Synthesis of 2-(1H-indol-3-yl)-N-(3-ethylbenzyl)-N-methylethan-1-amine

3-ethylbenzaldehyde (1 equiv.) was reacted with methylamine (1.2 equiv) at room temperature in ethanol overnight, followed by reductive amination using NaBH₄ (2.5 equiv) at room temperature for 2 h. After quenching excess hydride, the amine was recovered after extracting into aqueous acid, repeated washing with DCM, extraction back into DCM, washed with basic bicarbonate solution, before drying over K₂CO₃ and drying evaporation of solvent to give an oil (1 g, 91%). Coupling of the amine (1,2 equiv) with 3-indole acetic acid (1 equiv) was performed using polyphosphonic anhydride (2 equiv) and triethylamine (4 equiv) in acetonitrile at OC, which was allowed to naturally come to room temperature overnight. The workup included evaporation of solvent, redissolution in DCM, washing with mild aqueous acid and base to remove starting materials, drying over K₂CO₃ and evaporation of the solvent to yield a semisold (1.1 g, 54%), which was then reduced with LIAlH₄ (2 equiv) in THF at OC over 4 h. After reduction of the solvent, the compound was separated by flash chromatography EtOAc/heptane gradient to yield the desired compound as a semisolid (0.26 g, 29%). Purity (UPLC: 95.9%). MS MH+293. 1H-NMR (dmso-d6): 1.17 (t, 3H), 2.23 (s, 3H), 2.6 (m-m, 4H), 2.87 (t, 2H), 3.52 (s, 2H), 6.9 (t, 1H), 7.0-7.17 (mm, 5H), 7.2 (t, 1H), 7.3 (t, 1H), 7.4 (t, 1H), 10.75 (br s, 1H).

### Example 22—3-(((2-(1H-indol-3-yl)ethyl)(methyl)amino)methyl)phenol

The following reaction scheme was used:

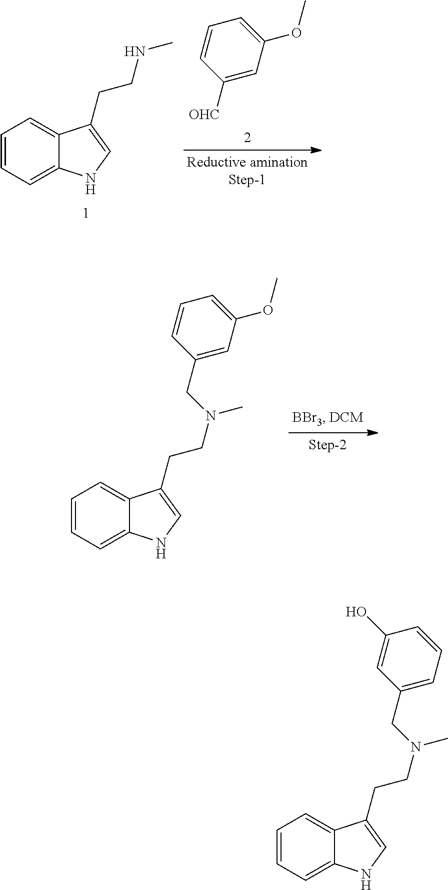

To a stirred solution of 2-(1H-indol-3-yl)-N-methylethan-1-amine (0.7 g, 1.0 equiv) in 25 ml of DCE were added 3-methoxybenzaldehyde (0.6 g, 1.0 equiv) and NaBH(OAc)3 (1.3 g, 1.5 equiv). The reaction mixture was stirred at room temperature for 16 h. The progress of reaction was monitored by TLC. After completion, the reaction mixture was diluted with DCM and quenched with saturated NaHCO₃ solution. The organic layer was separated, and aqueous layer was extracted with DCM (30 ml×2). Then organic layer was washed with brine solution and dried over anhydrous Na₂SO₄, and filtered, concentrated in vacuo. The crude was purified by Flash chromatography (Combi-Flash Column, 24 g redisep cartridge) using 100% DCM to 2% MeOH in DCM to get desired product as thick orange syrup (0.5 g, 44% yield).

To a stirred solution of 2-(1H-indol-3-yl)-N-(3-methoxybenzyl)-N-methylethan-1-amine (0.5 g, 1.0 equiv) in 25 ml of DCM was added BBr₃ (0.5 ml, 3.0 equiv) dropwise at 0° C. The reaction mixture was stirred at room temperature for 16 h. The progress of reaction was monitored by TLC. After completion, the reaction mixture was quenched with saturated NaHCO₃ solution and aqueous layer was extracted with DCM (50 ml×2). Then organic layer was washed with brine solution and dried over anhydrous Na₂SO₄, and filtered, concentrated in vacuo. The crude was purified by Flash chromatography (Combi-Flash Column, 24 g redisep cartridge) using 100% DCM to 5% MeOH in DCM and washed with n-heptane to get desired product as off white solid (0.1 g, 25% yield).

MS (ESI) m/e [M+H]⁺: 281; HPLC purity: 97.5% (RT=7.9 min), ¹H NMR (400 MHz, DMSO-d₆) δ=10.76 (s, 1H, indoleNH), 9.28 (s, 1H, aromOH), 7.44 (d, J=7.9 Hz, 1H, aromCH), 7.32 (d, J=7.9 Hz, 1H, aromCH), 7.28-6.98 (m, 3H, aromCH), 6.97-6.90 (m, 1H, aromCH), 6.88-6.71 (m, 2H, aromCH), 6.65 (d, J=6.1 Hz, I H, aromCH), 3.52 (s, 2H, CH2), 2.90 (s, 2H, CH2), 2.67 (s, 2H, CH2), 2.27 (s, 3H, NMe).

### Example 23—Synthesis of N-(3-bromobenzyl)-2-(1H-indol-3-yl)-N-methylethan-1-amine

The following reaction scheme was used:

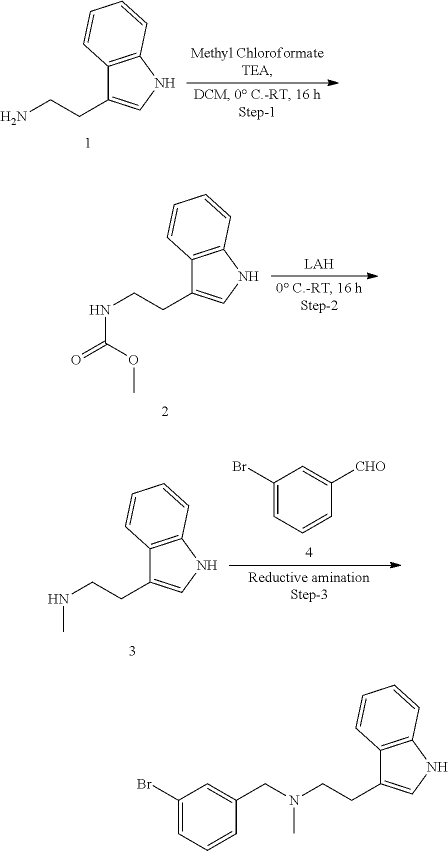 [SMILES: NCCc1c[nH]c2ccccc12 ; COC(=O)NCCc1c[nH]c2ccccc12 ; CNCCc1c[nH]c2ccccc12 ; O=Cc1cccc(Br)c1 ; CN(CCc1c[nH]c2ccccc12)Cc1cccc(Br)c1]

To a stirred solution of 2-(1H-indol-3-yl)ethan-1-amine (5 g, 1.0 equiv) in 50 ml of DCM, were added Et₃N (13 ml, 3.0 equiv) and methyl chloroformate (2.9 ml, 1.2 equiv) at 0° C. The reaction mixture was stirred at RT for 16 h. The progress of reaction was monitored by TLC. After completion, the reaction was quenched with ice-water and aqueous layer was extracted with DCM (50 ml×2). The organic layer was washed with brine solution, dried over anhydrous Na₂SO₄, and filtered, concentrated in vacuo. The crude was purified by Flash chromatography (Combi-Flash Column, 24 g redisep cartridge) using 30% of ethyl acetate in n-heptane to get desired product as off white solid (4.8 g, 70% yield).

To a stirred solution of N-(2-(1H-indol-3-yl)ethyl)propionamide (4.8 g, 1.0 equiv) in 100 ml of THF was added 2M solution of LAH (3.9 ml, 3.0 equiv) dropwise at 0° C. The reaction mixture was stirred at room temperature for 16 h. The progress of reaction was monitored by TLC. After completion, the reaction mixture was quenched with a saturated Na₂SO₄ solution (10 mL), white precipitate was filtered through pad of Celite and washed with EtOAc (100 mL). Filtrate was washed with water (30 mL), dried over anhydrous Na₂SO₄, filtered and concentrated in vacuo. The crude obtained was purified by combi flash chromatography to afford 2-(1H-indol-3-yl)-N-methylethan-1-amine (3.2 g, 82% yield) as thick orange syrup.

To a stirred solution of 2-(1H-indol-3-yl)-N-methylethan-1-amine (1.0 g, 1.0 equiv) in 25 ml of DCE were added 3-bromobenzaldehyde (1.3 g, 1.2 equiv) and NaBH(OAc)₃ (1.8 g, 1.5 equiv) at RT. The Reaction mixture was stirred at room temperature for 16 h. The progress of reaction was monitored by TLC. After completion, the RM was diluted with DCM and quenched with saturated NaHCO₃ solution. The organic layer was separated and washed with water followed by brine solution and dried over anhydrous Na₂SO₄, and filtered, concentrated in vacuo. The crude was purified by Flash chromatography (Combi-Flash Column, 24 g redisep cartridge) using 10-40% of ethyl acetate in n-heptane to get desired product as thick orange syrup (0.6 g, 30% yield).

MS (ESI) m/e [M+H]⁺: 343; HPLC purity: 98.6% (RT=5.4 min), ¹H NMR (400 MHz, DMSO-d₆) δ=10.75 (s, 1H, indoleNH), 7.51 (s, 1H, aromCH), 7.43 (d, J=7.9 Hz, 2H, aromCH), 7.37-7.17 (m, 3H, aromCH), 7.12 (s, 1H, aromCH), 7.04 (t, =7.5 Hz, I H, aromCH), 6.94 (t, J=8.0 Hz 1H, aromCH), 3.56 (s, 2H, CH2), 2.88 (t, J=8.0 Hz, 2H, CH2), 2.63 (t, J=8.0 Hz, 2H, CH2), 2.24 (s, 3H, NMe).

### Example 24—Synthesis of N-(2-(1H-indol-3-yl)ethyl)-N-(3-hydroxymethylbenzyl)methan-1-amine

To a stirred solution of 2-1H-indol-3-yl)-N-methylethan-1-amine (0.9 g, 1.2 equiv) and isophthalic acid monomethyl ester (1.0 equiv) and triethylamine (4 equiv) in 25 ml of ACN at OC was added polyphosphonic anhydride (2 equiv) and the reaction was allowed to come to RT gradually over 16 h. Progress of the reaction was monitored by TLC. After completion, the reaction mixture was concentrated in vacuo, diluted with water (30 mL), extracted with EtOAc (2×50 mL). Separated organic layer was dried over anhydrous Na2SO4, filtered and concentrated in vacuo. The crude obtained was purified by combi flash chromatography to get the desired product as an off-white solid (1.3 g, 76%). In a second step, the solid was reduced with LiAlH4 (3 equiv) in 30 ml THE at reflux for 16 h. The progress of reaction was monitored by TLC. After completion, the reaction mixture was quenched with a saturated Na₂SO₄ solution (10 mL), white precipitate was filtered through pad of Celite and washed with EtOAc (100 mL). Filtrate was washed with water (30 mL), dried over anhydrous Na₂SO₄, filtered and concentrated in vacuo. The crude obtained was purified by combi flash chromatography to afford 2-(1H-indol-3-yl)-N-(3-hydroxymethylbenzyl)-N-methylethan-1-amine as a semi-solid (0.35 g, 30%).

MS (ESI) m/e [MH+]295. HPLC purity: 98% (RT=7.56 min), ¹H NMR (400 MHz, DMSO-d₆) δ=2.2 (br, 2H), 2.4 (s, 3H), 2.85 (t, 2H), 3.08 (t, 2H), 3.7 (s, 2H), 4.7 (s, 2H), 7.05 (s, 1H), 7.13 (t, 1H), 7.22 (t, 1H), 7.29-7.4 (overlapping multiplets, 5H), 7.56 (d, 1H), 8.1 (br s, 1H).

### Example 25—Synthesis of 2-(1H-indol-3-yl)-N-(3-ethynylbenzyl)-N-methylethan-1-amine

To an ethanolic solution containing 2-(1H-indol-3-yl)-N-methylethan-1-amine (0.5 g, 1.2 equiv, as prepared in Example 23) and 3-ethynyl-benzaldehyde (1.1 equiv), which had been stirred at room temperature overnight was added NaBH₄ (2.5 equiv). The mixture was stirred an additional 2 h before workup. The reaction mixture was quenched with ice-water and extracted with ethyl acetate (50 ml×2). The organic layer was washed with brine solution and dried over anhydrous Na₂SO₄, and filtered, concentrated in vacuo. The crude was purified by Flash chromatography (Combi-Flash Column, 24 g redisep cartridge) using 100/a DCM to 2% MeOH in DCM to get desired product as thick orange syrup (0.3 g, 22% yield). MS (ESI) m/e [MH+]289.2. Purity HPLC 99.56%. ¹H NMR (400 MHz, DMSO-d₆): structure conforms.

### Example 26—Synthesis of 241H-indol-3-yl)-N-(2,3-methylenedioxybenzyl)-N-methylethan-1-amine

To a stirred solution of 2-(1H-indol-3-yl)-N-methylethan-1-amine (0.5 g, 1.2 equiv, prepared in Example 23) in 25 ml of acetonitrile was added 2,3-methylenedioxy-benzoic acid (1.0 equiv), polyphosphonic anhydride (1.5 equiv) and triethylamine (3 equiv) at 0° C. and the reaction was allowed ot come to room temperature with stirring overnight. The progress of reaction was monitored by TLC. After completion, the RM was diluted with DCM and quenched with saturated NaHCO₃ solution. The organic layer was separated and washed with water followed by brine solution and dried over anhydrous Na₂SO₄, and filtered, concentrated in vacuo. The crude was purified by Flash chromatography (Combi-Flash Column, 24 g redisep cartridge) using 10-40% of ethyl acetate in n-heptane to get desired product (0.7 g, 69% yield). The resultant semisolid was then reduced using 2 equiv of LiAlH₄ in THF at OC over 16 h to yield the desired product which was again purified by flash chromatography (0.3 g, 42%). Purity (HPLC): 98.7%. MS (ESI) m/e MH+309. 1H-NMR (dmso-d6): 2.25 (3H, Me), 2.65 (m, 2H), 2.87 (m, 2H), 3.55 (s, 2H), 6.0 (s, 2H), 6.8 (m-m, 3H), 6.95 (t, (1H), 7.05 (t, 1H), 7.13 (m, 1H), 7.3 (d, 1H), 7.45 (d, 1H), 10.75 (br s, 1H).

### Example 27-Synthesis of 2-(5-methoxy-1H-indol-3-yl)-N-(3-methoxybenzyl)-N-methylethan-1-amine

The following reaction scheme was used:

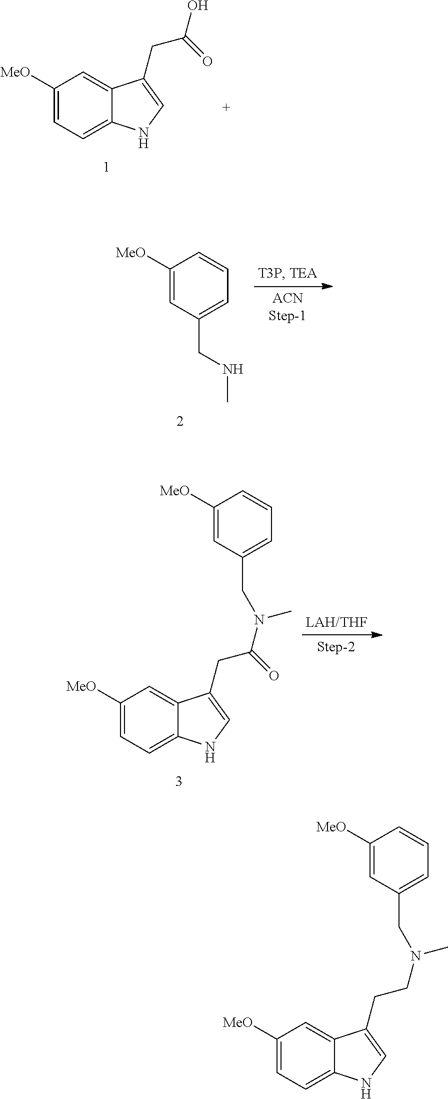 [SMILES: COc1ccc2[nH]cc(CC(=O)O)c2c1 ; CNCc1cccc(OC)c1 ; COc1cccc(CN(C)C(=O)Cc2c[nH]c3ccc(OC)cc23)c1 ; COc1cccc(CN(C)CCc2c[nH]c3ccc(OC)cc23)c1]

To a stirred solution of 2-(5-methoxy-1H-indol-3-yl)acetic acid (0.5 g, 1.0 equiv) in ACN were added 1-(3-methoxyphenyl)-N-methylmethanamine (0.4 g, 1.2 equiv) followed by T₃P (1.6 g, 2.0 equiv) and Et₃N (0.8 g, 3.3 equiv) at room temperature. The reaction mixture was stirred at room temperature for 16 h. Progress of the reaction was monitored by TLC. After completion, the reaction mixture was concentrated in vacuo, diluted with water (30 mL), extracted with EtOAc (2×50 mL). Separated organic layer was dried over anhydrous Na₂SO₄, filtered and concentrated in vacuo. The crude obtained was purified by combi flash chromatography (Combi-Flash Column, 24 g redisep cartridge) using 5% of DCM:MeOH to get the product as brown colour solid (0.62 g, 75% yield).

To a stirred solution of 2-(5-methoxy-1H-indol-3-yl)-N-(3-methoxybenzyl)-N-methylacetamide (0.62 g, 1.0 equiv) in THF was added a solution of 2M LAH (0.14 g, 2.0 equiv) dropwise. The reaction mixture was stirred at 0° C. for 4 h under N₂ atmosphere. Progress of the reaction was monitored by TLC. After completion, the reaction mixture was quenched with a saturated Na₂SO₄ solution (10 mL), white precipitate was filtered through pad of Celite and washed with EtOAc (100 mL). Filtrate was washed with water (30 mL), dried over anhydrous Na₂SO₄, filtered and concentrated in vacuo. The crude obtained was purified by combi flash chromatography to afford 2-(5-methoxy-1H-indol-3-yl)-N-(3-methoxybenzyl)-N-methylethan-1-amine (FT230, 0.35 g, 60%) as an off white solid.

MS (ESI) m/e [M+H]⁺: 325; HPLC purity: 96.44% (RT=4.0 min), ¹H NMR (400 MHz, DMSO-d₆) δ=10.59 (s, 1H, indoleNH), 7.27-7.12 (m, 2H, aromCH), 7.08 (d, J=2.0 Hz, 1H, aromCH), 6.88 (d, J=5.4 Hz, 3H, aromCH), 6.80 (d, J=7.8 Hz, 1H, aromCH), 6.68 (dd, J=2.4, 8.8 Hz, 1H, aromCH), 3.70 (d, J=2.0 Hz, 6H, OMe), 3.53 (s, 2H, CH2), 2.84 (t, J=4.0 Hz 2H, CH2), 2.62 (s, 2H, CH2), 2.26 (s, 3H, NMe).

### Example 28—Synthesis of 2-(1H-indol-3-yl)-N-(2-methoxybenzyl)-N-methylethan-1-amine

The following reaction scheme was used:

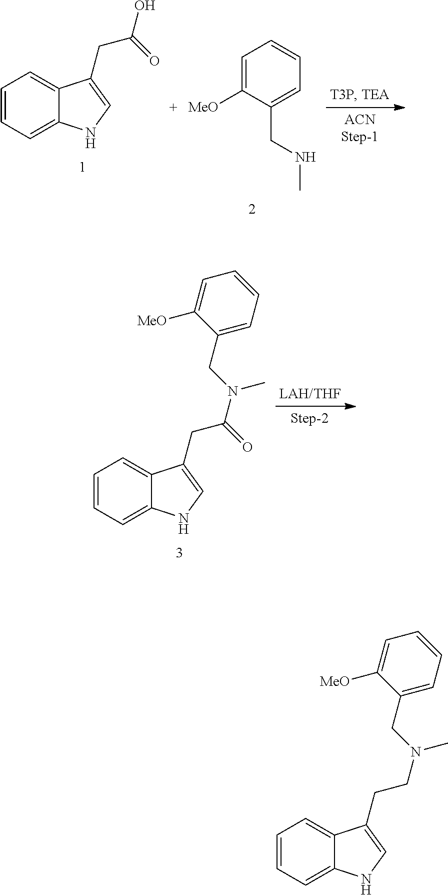 [SMILES: O=C(O)Cc1c[nH]c2ccccc12 ; CNCc1ccccc1OC ; COc1ccccc1CN(C)C(=O)Cc1c[nH]c2ccccc12 ; COc1ccccc1CN(C)CCc1c[nH]c2ccccc12]

To a stirred solution of 2-(1H-indol-3-yl)acetic acid (1 g, 1.0 equiv) in ACN solvent were added 1-(2-methoxyphenyl)-N-methylmethanamine (1.6 g, 2.0 equiv) followed by T₃P (3.6 g, 2.0 equiv) and Et₃N (1.7 g, 3.0 equiv) at room temperature. The reaction mixture was stirred at RT for 16 h. Progress of the reaction was monitored by TLC. After completion, the reaction mixture was concentrated in vacuo, diluted with water (30 mL), extracted with EtOAc (2×50 mL). Separated organic layer was dried over anhydrous Na2SO4, filtered and concentrated in vacuo. The crude obtained was purified by combi flash chromatography to get the desired product (1.2 g, 68% yield).

To a stirred solution of 2-(1H-indol-3-yl)-N-(2-methoxybenzyl)-N-methylacetamide (1.2 g, 1.0 equiv) in THF was added a solution of 2M LAH (0.3 g, 2.0 equiv) solution dropwise. The reaction mixture was stirred at 0° C. for 4 h under N2 atmosphere. Progress of the reaction was monitored by TLC. After completion, the reaction mixture was quenched with a saturated Na2SO4 solution (10 mL), white precipitate was filtered through pad of Celite and washed with EtOAc (100 mL). Filtrate was washed with water (30 mL), dried over anhydrous Na2SO4, filtered and concentrated in vacuo. The crude obtained was purified by combi flash chromatography to afford 2-(1H-indol-3-yl)-N-(2-methoxybenzyl)-N-methylethan-1-amine (FT231, 0.31 g, 27%) as an off white solid.

MS (ESI) m/e [M+H]⁺: 295; HPLC purity: 98% (RT=5.9 min), ¹H NMR (400 MHz, DMSO-d₆) 5=10.74 (s, 1H, indoleNH), 7.44 (d, 0.1=8.0 Hz, 1H, aromCH), 7.36-7.29 (m, 2H, aromCH), 7.25-7.17 (m, 1H, aromCH), 7.12 (d, J=2.3 Hz, 1H, aromCH), 7.08-7.01 (m, 1H, aromCH), 6.99-6.86 (m, 3H, aromCH), 3.76 (s, 3H, OMe), 3.55 (s, 2H, CH2), 2.88 (s, J=4.0 Hz 2H, CH2), 2.65 (s, J=8.0 Hz, 2H, CH2), 2.26 (s, 3H, NMe).

### Example 29—Synthesis of N-(2-(1H-indol-3-yl)ethyl)-N-(3-methoxybenzyl)prop-2-en-1-amine

The following reaction scheme was used:

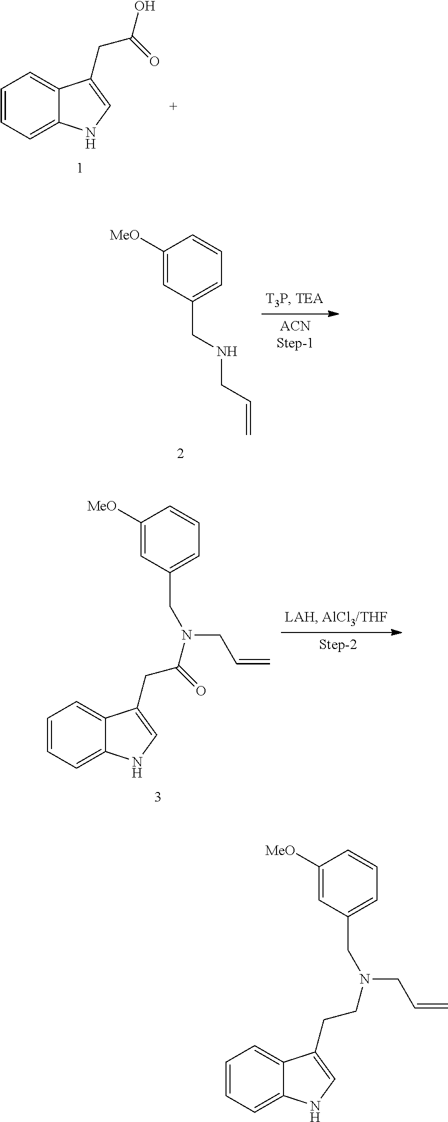 [SMILES: O=C(O)Cc1c[nH]c2ccccc12 ; C=CCNCc1cccc(OC)c1 ; C=CCN(Cc1cccc(OC)c1)C(=O)Cc1c[nH]c2ccccc12 ; C=CCN(CCc1c[nH]c2ccccc12)Cc1cccc(OC)c1]

To a stirred solution of 2-(1H-indol-3-yl)acetic acid (0.6 g, 1.0 equiv) in 10 ml of ACN solvent were added N-(3-methoxybenzyl)prop-2-en-1-amine (0.7 g, 1.2 equiv) followed by T₃P (2.2 g, 2.0 equiv) and Et₃N (1.0 g, 3.0 equiv) at RT. The reaction mixture was stirred at RT for 16 h under N₂ atmosphere. After completion, the reaction mixture was concentrated under reduced pressure and diluted with DCM and extracted with DCM and water (40 ml). The organic layer was dried over anhydrous Na₂SO₄ and concentrated in vacuo. The crude was purified by combi flash chromatography to get the desired product as off white solid (1.1 g, 96% yield).

To a stirred solution of LAH (0.5 g, 4.0 equiv) in 10 ml of THF was added AlCl₃ (1.9 g, 4.0 equiv) portionwise. Then the reaction mixture was continuously stirred at 0° C. to RT for 1 hr. After 1 hr, N-allyl-2-(1H-indol-3-yl)-N-(3-methoxybenzyl)acetamide (1.1 g, 1.0 equiv) in THF was added dropwise and the reaction mixture was stirred at RT for 12 h. After completion, the reaction mixture was quenched with a saturated Na₂SO₄ solution (10 mL), white precipitate was filtered through pad of Celite and washed with EtOAc (100 mL). Filtrate was washed with water (30 mL), dried over anhydrous Na₂SO₄, filtered and concentrated in vacuo. The crude obtained was purified by combi flash chromatography to afford N-(2-(1H-indol-3-yl)ethyl)-N-(3-methoxybenzyl)prop-2-en-1-amine (FT233, 0.6 g, 59/a) as a brown colour solid.

MS (ESI) m/e [M+H]⁺: 321; HPLC purity: 99/a (RT=5.8 min), ¹H NMR (400 MHz, DMSO-d₆) δ=10.73 (s, 1H, indoleNH), 7.38 (d, J=7.75 Hz, 1H, aromCH), 7.3 (d, J=8.0 Hz, 1H, aromCH), 7.22 (t, J=7.88 Hz, 1H, aromCH), 7.11-6.98 (m, 2H, aromCH), 6.91 (s, 3H, aromCH), 6.8 (d, J=7.5 Hz, 1H, aromCH), 6.00-5.80 (m, l H, alkeneCH), 5.24 (d, J=17.3 Hz, 1H, alkeneCH), 5.15 (d, J=10.26 Hz, 1H, alkeneCH), 3.71 (s, 3H, OMe), 3.63 (s, 2H, CH2), 3.17 (d, J=5.75 Hz, 2H, CH2), 2.86 (t, J=8.0 Hz, 2H, CH2), 2.69 (t, J=8.0 Hz, 2H, CH2).

### Comparative Examples A-C

Using a methodology similar to that described above for the compounds of Examples 1 and 8, the following compounds were synthesized and tested:

| Example | Compound |
|---|---|
| A | 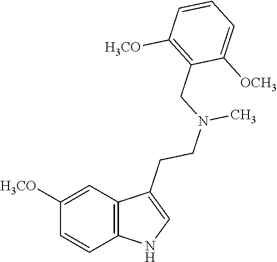 [SMILES: COc1ccc2[nH]cc(CCN(C)Cc3c(OC)cccc3OC)c2c1] |
| B | 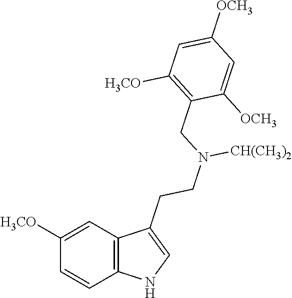 [SMILES: COc1cc(OC)c(CN(CCc2c[nH]c3ccc(OC)cc23)C(C)C)c(OC)c1] |
| C | 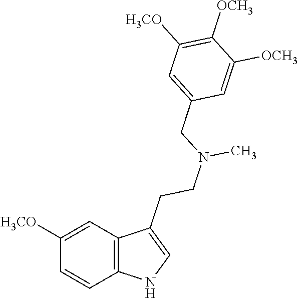 [SMILES: COc1ccc2[nH]cc(CCN(C)Cc3cc(OC)c(OC)c(OC)c3)c2c1] |

The compounds of Comparative Examples A-C are outside the scope of the present invention. The results are reported in Table 1.

TABLE 1

| | | | 2B IC50 | 2B EC50 |
|---|---|---|---|---|
| | | | Functional | Functional |
| Example | 2A Binding | 2B Binding | Antagonist | Agonist |
| 11 | 170 | 173 | — | >10,000 |
| 12 | 277 | 341 | — | >10,000 |
| 13 | 76 | 73 | >10,000 | >10,000 |
| 14 | 280 | 69 | 1670 | — |
| 15 | 274 | 48 | 1370 | — |
| 16 | 2630 | 313 | >10,000 | >10,000 |
| 17 | 60 | 45 | >10,000 | — |
| 18 | 16 | — | 2840 | — |
| 19 | 31 | 44 | 3380 | >10,000 |
| 20 | 135 | — | — | — |
| 21 | 5 | — | 687 | — |
| 22 | 35 | 31 | >10,000 | — |
| 23 | 11 | 22 | 1620 | — |
| 24 | 59 | 50 | 6990 | — |
| 25 | 6 | 13 | 2560 | 163 |
| 26 | 73 | 60 | 1090 | — |
| 27 | 16 | 16 | — | — |
| 28 | 130 | 253 | — | — |
| 29 | 28 | 21 | — | — |
| A | 1600 | 402 | >10,000 | — |
| B | 4600 | 4930 | >10,000 | — |
| C | 340 | 76 | 2050 | — |

Thus, serotonin-2A agonist has been tested and is proposed to treat multiple mood disorders by a new mechanism of action and thus could rescue patients not treatable with current medical options. However, agonists of serotonin-2A are often agonists of the nearly homologous serotonin-2B receptor. As mentioned above, agonism of the serotonin-2B receptor can result in valve hardening by a fibrotic mechanism, and was exemplified by the compound fenfluramine which was withdrawn from the pharmaceutical weight-loss market due to this adverse reaction in overweight patients. The present invention provides for molecules, methods and uses of new molecules based on benzyl-functionalized tryptamine that demonstrate strong serotonin-2A receptor agonism, and are atypically serotonin-2B antagonists and thus do not present cardiotoxic adverse reaction potential. The present invention could find use in novel mood indications, neural restoration/repair (neuroplasticity) and potential as more frequently used or chronic daily medicines for patients. Further the invention may allow patients with pre-existing cardiovascular fragilities to use these molecules after appropriate demonstrations of efficacy and safety to treat these disorders of the brain and who would otherwise be excluded from the use of classical serotonin agonists that more traditionally activate serotonin-2B.

While this invention has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications or embodiments.

All publications, patents and patent applications referred to herein are incorporated by reference in their entirety to the same extent as if each individual publication, patent or patent application was specifically and individually indicated to be incorporated by reference in its entirety.

1-24. (canceled)

25. A compound, wherein the compound is represented by Formula I or is a pharmaceutically acceptable salt thereof: 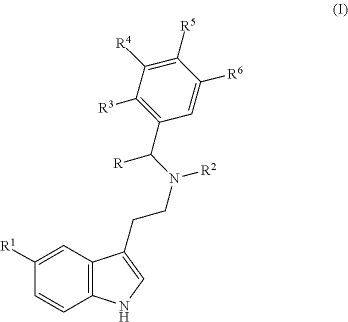
wherein:
R is methyl or ethyl;
R¹ is hydrogen;
R² is methyl; and
R³, R⁴, R⁵, and R⁶ are each hydrogen.

26. A pharmaceutical composition comprising a compound and a pharmaceutically acceptable carrier, wherein the compound is represented by Formula I or is a pharmaceutically acceptable salt thereof: 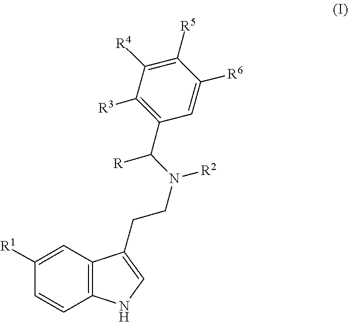
wherein:
R is methyl or ethyl;
R¹ is hydrogen;
R² is methyl; and
one adjacent pair selected from R³, R⁴, R⁵, and R⁶ join together to form a ring having at least 5 members, and the remaining two of R³, R⁴, R⁵, and R⁶ are hydrogen.

27. A method of treating a central nervous system (CNS) disease or disorder in a subject in need thereof, the method comprising administering to the subject a therapeutically effective amount of a compound, wherein the compound is represented by Formula I or is a pharmaceutically acceptable salt thereof: 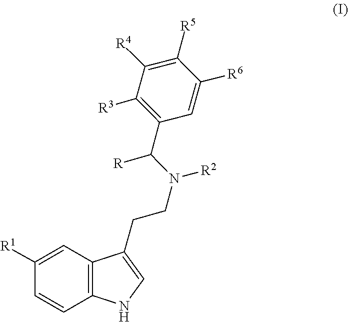
wherein:
R is hydrogen;
R¹ is C₁-C₂ alkoxy;
R² is a C₂-C₄ group which may be saturated or unsaturated, branched or linear; and
two of R³, R⁴, R, and R⁶ are each independently selected from, hydroxyl, halogen, methyl optionally substituted with hydroxy, methoxy, ethoxy, and a saturated or unsaturated C₂-C₃ that may be optionally substituted with hydroxyl, and the remaining two of R³, R⁴, R⁵, and R⁶ are hydrogen.
